(19) United States
(12) Patent Application Publication
(10) Pub. No.: US 2016/0287602 A1
(43) Pub. Date: Oct. 6, 2016

(54) METHODS FOR PROMOTING MOTOR NEURON SURVIVAL

(71) Applicant: PRESIDENT AND FELLOWS OF HARVARD COLLEGE, Cambridge, MA (US)

(72) Inventors: Lee L. Rubin, Wellesley, MA (US); Shailesh K. Gupta, Somerville, MA (US)

(21) Appl. No.: 15/035,369

(22) PCT Filed: Nov. 7, 2014
(86) PCT No.: PCT/US14/64638

§ 371 Date: May 9, 2016

Related U.S. Application Data
(60) Provisional application No. 61/901,926, filed on Nov. 8, 2013.

(51) Int. Cl. A61K 31/5377; C12Q 1/68; G01N 33/50; G01N 33/573; A61K 31/517; A61K 31/506

(52) U.S. Cl. CPC A61K 31/5377; A61K 31/517; A61K 31/506; G01N 33/5058; G01N 33/573; C12Q 1/6883; G01N 2333/912; G01N 2800/28; G01N 2800/52; C12Q 2600/158; C12Q 2600/106; C12Q 2600/112; C12Q 2600/156

(57) ABSTRACT

The present invention relates to methods for promoting motor neuron survival, treating or preventing neurodegenerative disorders, identifying agents that promote survival of motor neurons, identifying agents that are useful for treating neurodegenerative disorders, diagnosing neurodegenerative disorders, predicting the progression of a neurodegenerative disorder in a subject, and monitoring the effectiveness of a therapy in reducing the progression of a neurodegenerative disorder in a subject.

### RELATED APPLICATIONS

This application claims the benefit of U.S. Provisional Application No. 61/901,926, filed on Nov. 8, 2013. The entire teachings of the above application are incorporated herein by reference.

### GOVERNMENT SUPPORT

This invention was made with government support under 5P01NS066888-02 awarded by the National Institutes of Health. The government has certain rights in the invention.

### BACKGROUND OF THE INVENTION

Amyotrophic lateral sclerosis (ALS) is a late-onset, progressive, neurodegenerative disorder that affects motor neuron survival of both the upper and lower motor neurons (MNs) and ultimately leads to death. Although the rate at which ALS progresses can be quite variable, the mean survival time is between three and five years. In the United States, 90 to 95% of all ALS cases are idiopathic (Brown, 1997; Boillée et al., 2006). However, among the familial forms of ALS, approximately 20% are caused by mutations in the SOD1 gene. Although only accounting for about 2% of all ALS cases, SOD1-associated ALS has been the most studied form of ALS, due to the early discovery of the disease-causing mutations and the availability of mouse models. Mutations in SOD1 gene are gain-of-function mutations that cause autosomal dominant inheritance of ALS. It is the toxicity of the mutant SOD1 protein, rather than a defect in the function of the normal SOD1 protein, that is thought to lead to the disease. Exactly how mutations in the SOD1 gene cause MN death is still unclear, but it is now well accepted that cell autonomous and non-cell autonomous mechanisms can contribute to degeneration (Di Giorgio et al., 2007; Nagai et al., 2007; reviewed in Ilieva et al., 2009). A more recent breakthrough in ALS research came when the DNA/RNA-binding protein transactivating response element DNA binding protein-43 (TDP-43) was identified as a major component of protein aggregates found in sporadic ALS and non-SOD1 familial ALS cases (Arai et al., 2006; Neumann et al., 2006). Later, mutations in TARDBP, the gene encoding TDP-43, were identified in ˜4% of familial ALS cases (Van Deerlin et al., 2008). The very recent identification of a hexanucleotide repeat expansion within the C9orf72 gene points to it as potentially the most frequent pathogenic cause of ALS identified thus far, accounting overall for about 6% of sporadic ALS cases, and about 3˜40% of familial ALS cases, in Europe and the USA (Renton et al., 2011; Majounie et al., 2012). Thus, it may be that there are numerous pathogenic initiators of ALS, potentially including mitochondrial dysfunction, oxidative stress, protein misfolding and aggregation, excitotoxicity, neuroinflammation, axonal transport defects, and neurotrophin depletion (Joyce et al., 2011). Riluzole is currently the only approved treatment for ALS. It may act by reducing an excitotoxic component of the disease, but it prolongs life by only 2 to 3 months and provides little functional improvement (Miller et al., 2007). While better treatments for ALS are urgently needed, it has been challenging to conduct research geared towards therapeutic discovery, partly because of the diverse causes of ALS. Therefore, there is need in the art for methods of identifying agents for promoting motor neuron survival and methods for treatment of motor neuron diseases such as ALS and SMA.

### SUMMARY OF THE INVENTION

Disclosed herein are one or more solutions to the needs outlined above.

In an aspect, the present invention provides a method of promoting motor neuron survival, comprising contacting a motor neuron or population of cells comprising a motor neuron with an effective amount of an agent that inhibits Aurora kinase.

In an aspect, the present invention provides a method of treating or preventing a neurodegenerative disorder in a subject in need thereof, comprising administering to the subject an effective amount of an agent that inhibits Aurora kinase.

In some embodiments, the agent increases activation of the anti-apoptotic protein kinase A pathway. In some embodiments, the agent increases phosphorylation of a protein in the anti-apoptotic protein kinase A pathway. In some embodiments, the protein is Bc1-2-associated death promoter (BAD). In some embodiments, the agent is a pan Aurora kinase inhibitor. In some embodiments, the agent inhibits Aurora kinase A. In some embodiments, the agent inhibits Aurora kinase B. In some embodiments, the agent inhibits Aurora kinase C. In some embodiments, the agent is selected from the group consisting of VX-608, ZM447439, 4-4-Ben, MLN8054, PHA-680632, TAK-901, AMG900, PF-03814735, CCT129202, phtalazinonepyrazole, hesperidin hydrochloride, CCT 137690, TC-A 2317 hydrochloride, AurkA III, Aurora kinase inhibitor II, JNJ-7706621, H-1152, PHA739358, OM137, SNS-314, AT9283, CYC-116, MLN8237, ENMD-2076, SBE 13 hydrochloride, analogs or derivatives thereof, and combinations thereof. In some embodiments, the agent is selected from the group consisting of small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

In some embodiments, the motor neuron are selected from the group consisting of a HB9 motor neuron, a G93A motor neuron, a HB9(WT-SOD1) motor neuron, a HUES3 derived motor neuron, and combinations thereof.

In some embodiments, the motor neuron comprises a mutation in a gene encoding superoxide dismutase 1 (SOD1). In some embodiments, the mutation is a G93A mutation. In some embodiments, the contact is in vitro or ex vivo.

In some embodiments, the subject selected for treatment of a neurodegenerative disorder or disorder characterized by neuronal cell death. In some embodiments, the subject is at risk of developing a neurodegenerative disorder or a disorder characterized by neuronal cell death. In some embodiments, the subject is suspected of having a neurodegenerative disorder or a disorder characterized by neuronal cell death. In some embodiments, the subject is a mammal. In some embodiments, the subject is a human.

In some embodiments, the neurodegenerative disorder is characterized by mutation of a SOD gene. In some embodiments, the neurodegenerative disorder is characterized by decreased levels of SOD protein. In some embodiments, the neurodegenerative disorder is characterized by neuronal cell death. In some embodiments, the neurodegenerative disorder is ALS.

In an aspect, the present invention provides a method of identifying a candidate agent that promotes motor neuron survival, comprising (a) contacting a population of cells comprising motor neurons with a test agent, and (b) measuring (i) the level or activity of an Aurora kinase or (ii) activation of the anti-apoptotic protein kinase A pathway, in the presence of the test agent, and (c) identifying the candidate agent that promotes motor neuron survival, wherein the test agent is a candidate agent for promoting motor neuron survival if the test agent (i) decreases the level or activity of the Aurora kinase or (ii) increases activation of the anti-apoptotic protein kinase A pathway, in the presence of the test agent.

In an aspect, the present invention provides a method of identifying a candidate agent for treating or preventing a neurodegenerative disorder, comprising (a) contacting a population of cells comprising motor neurons with a test agent, and (b) measuring (i) the level or activity of an Aurora kinase or (ii) activation of the anti-apoptotic protein kinase A pathway, in the presence of the test agent, and (c) identifying the candidate agent for treating or preventing a neurodegenerative disorder, wherein the test agent is a candidate agent for treating or preventing a neurodegenerative disorder if the test agent (i) decreases the level or activity of the Aurora kinase or (ii) increases activation of the anti-apoptotic protein kinase A pathway, in the presence of the test agent.

In some embodiments, the neurodegenerative disorder is amyotrophic lateral sclerosis. In some embodiments, the population of cells comprises glial cells. In some embodiments, the population of cells comprises astrocytes. In some embodiments, the contacting is performed in the absence of trophic factors. In some embodiments, the motor neurons are selected from the group consisting of a HB9 motor neuron, a G93A motor neuron, a HB9(WT-SOD1) motor neuron, a HUES3 derived motor neuron, and combinations thereof. In some embodiments, the motor neuron comprises a mutation in a gene encoding superoxide dismutase 1 (SOD1). In some embodiments, the mutation is a G93A mutation. In some embodiments, the motor neuron comprises an in vitro-differentiated motor neuron. In some embodiments, the motor neurons are derived from pluripotent cells selected from the group consisting of embryonic stem cells (ESCs) and induced pluripotent stem cells (iPSCs). In some embodiments, the motor neurons are derived from an individual suffering from, diagnosed with, or at risk of developing ALS. In some embodiments, the motor neurons comprise human motor neurons.

In some embodiments, the test agent is selected from the group consisting of small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

In some embodiments, the method comprises quantifying the number of motor neurons surviving in the presence of the test agent. In some embodiments, the surviving motor neurons express a detectable reporter. In some embodiments, the detectable reporter is a fluorescent protein selected from the group consisting of green fluorescent protein (GFP) and red fluorescent protein (RFP).

In an aspect, the present invention provides a method for diagnosing a neurodegenerative disorder in a subject, the method comprising: (a) obtaining a biological sample comprising neuronal cells from the subject; (b) conducting at least one assay on the neuronal cells in the biological sample to detect the level or activity of Aurora kinase in the neuronal cells; and (c) diagnosing the subject as having a neurodegenerative disorder if the level or activity of the Aurora kinase in the neuronal cells is increased relative to a level or activity of Aurora kinase in a control sample.

In an aspect, the present invention provides a method for predicting the progression of a neurodegenerative disorder in a subject, the method comprising: (a) obtaining a first biological sample comprising neuronal cells from a subject diagnosed as having a neurodegenerative disorder; (b) obtaining a second biological sample comprising neuronal cells from the subject at a time which is later than when the first biological sample was obtained; (c) conducting at least one assay on the neuronal cells in the biological samples to detect a level or activity of Aurora kinase in the neuronal cells; and (d) predicting the progression of the neurodegenerative disorder in the subject, wherein: (i) the neurodegenerative disorder is predicted to progress if the level or activity of Aurora kinase in the neuronal cells in the second biological sample is increased relative to the level or activity of Aurora kinase in in the first biological sample; or (ii) the neurodegenerative disorder is not predicted to progress if the level or activity of Aurora kinase in the neuronal cells in the second biological sample is decreased relative to the level or activity of Aurora kinase in in the first biological sample.

In an aspect, the present invention provides a method of monitoring the effectiveness of a therapy in reducing the progression of a neurodegenerative disorder in a subject, the method comprising: (a) conducting at least one assay to determine the level or activity of Aurora kinase in a biological sample comprising neuronal cells from a subject having a neurodegenerative disorder prior to and following administration of the therapy to the subject; and (b) comparing the level or activity of Aurora kinase in the biological sample from the subject prior to the administration of the therapy to the level or activity of Aurora kinase in the biological sample from the subject following administration of the therapy; and (c) monitoring the effectiveness of the therapy in reducing the progression of the neurodegenerative disorder in the subject, wherein a decrease in the level or activity of Aurora kinase in the biological sample following administration of the therapy as compared to the level or activity of Aurora kinase in the biological sample prior to the administration of the therapy is an indication that the therapy is effective in reducing the progression of the neurodegenerative disorder in the subject.

In some embodiments, the at least one assay comprises a hybridization assay to detect the expression of Aurora kinase. In some embodiments, the hybridization assay is selected from the group consisting of a microarray and qRT-PCR. In some embodiments, the at least one assay comprises a sequencing assay to detect the expression of Aurora kinase. In some embodiments, the sequencing assay is selected from the group consisting of serial analysis of gene expression (SAGE), cap analysis of gene expression (CAGE), massively parallel signature sequencing (MPSS), GRO-seq, and RNA-seq. In some embodiments, the at least one assay comprises immunostaining to detect Aurora kinase protein levels. In some embodiments, the immunostaining is selected from the group consisting of Western blot, ELISA, and flow cytometry. In some embodiments, the at least one assay comprises a phosphorylation assay to detect phosphorylation of Aurora kinase. In some embodiments, the at least one assay comprises a phosphorylation assay to detect phosphorylation of Aurora kinase at threonine 288 (T288) or serine 331 (S331). In some embodiments, the at least one assay comprises a phosphorylation assay to detect the phosphorylation activity of Aurora kinase. In some embodiments, the at least one assay comprises a protein kinase assay to detect the level of phosphorylation of a protein in the anti-apoptotic protein kinase A pathway. In some embodiments, the at least one assay comprises a protein kinase assay to detect the level of phosphorylation of BAD protein.

In some embodiments, the methods include selecting a subject suspected of having a neurodegenerative disorder.

In some embodiments, the neuronal cells comprise motor neurons. In some embodiments, the neuronal cells comprise sensory neurons. In some embodiments, the neurodegenerative disorder is ALS. In some embodiments, the Aurora kinase is selected from Aurora kinase A, Aurora kinase B, Aurora kinase C, and combinations thereof. In some embodiments, the therapy comprises an agent that inhibits Aurora kinase selected from a pan Aurora kinase inhibitor, an inhibitor of Aurora kinase A, an inhibitor of Aurora kinase B, and an inhibitor of Aurora kinase C.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A, 1B and 1C demonstrate that Aurora kinase inhibitor (AKIs) promote motor neuron survival. FIG. 1A is a schematic illustration of the directed differentiation of embryonic stem cells into motor neurons and screening flow. FIGS. 1B and 1C are line graphs depicting dose curves of AKIs on mixed and FACS purified motor neuron culture.

FIG. 2 demonstrates that Aurora kinase shRNA validates the target specificity of AKIs. Knockdown of all three subtypes of Aurora kinase significantly increased the survival of motor neurons, with type B showing the greatest effect.

FIGS. 3A and 3B demonstrate that Aurora kinase is activated in degenerating motor neurons. FIG. 3A is a Western blot showing an increase in activated (phosphorylated) Aurora kinase level in −TF Hb9:: GFP motor neuron culture. FIG. 3B depicts a Columbus software analysis for pAurk (red) and total Aurk (red) immunostaining in Hb9: GFP motor neuron culture.

FIGS. 4A and 4B demonstrate that AKIs preserve morphological integrity of surviving motor neurons. FIG. 4A is a Western blot showing an increase in expression of synaptic protein synaptophysin in AKIs treated motor neuron culture. FIG. 4B depicts a Columbus software analysis for synapsin (blue) and PSD95 (red) immunostaining in WT and SOD1^{G93A} mutant motor neuron culture.

FIGS. 5A and 5B demonstrate microarray analysis of gene expression in AKIs treated motor neurons. FIG. 5A illustrates that the results of microarray analysis performed on Hb9::GFP using Affymetrix 1.0 ST microarrays, depicting a column-centered heat map of hierarchical clustering carried out on the 1,075 differentially expressed genes at 2 fold cut off FIG. 5B is a Venn diagram analysis to identify overlapping genes within +TF, VX-680 and ZM447439 treated motor neurons. 725 overlapping genes were identified for Hb9:GFP motor neuron cultures.

FIGS. 6A, 6B and 6C demonstrate that AKIs promote motor neuron survival through the PKA pathway. FIG. 6A shows Prkarlb and pBAD average intensity per motor neuron per well analyzed using Columbus software. FIG. 6B shows that knocking down Prkarlb decreases motor neuron survival in the presence of TF, VX-680 and ZM447439. FIG. 6C is a diagrammatic illustration outlining the pathway that is active in the presence of AKIs or TF to promote motor neuron survival.

FIGS. 7A, 7B and 7C demonstrate the effect of AKIs on HuES-3/Hb9::GFP ESCs derived human motor neurons. FIG. 7A is a bar graph showing the fold increase in the survival of human motor neurons derived from Hues-3/Hb9:GFP ESCs. FIG. 7B is a bar graph showing ZM447439 decreases the toxic effect of SOD1^{G93A} astrocytes on HuES-3 human motor neurons in co-culture. FIG. 7C shows the size of human motor neurons analyzes using Columbus software.

FIG. 8A, 8B and 8C demonstrate that AKIs promote survival of human motor neurons derived from various ALS iPSCs. Bar graphs show the fold increase in the survival of human motor neurons derived from wild-type and SOD1^{L144F} (FIG. 8A), TDP-43^{M337V} and TDP-43^{G298S} (FIG. 8B), and C9orf72 iPSCs (FIG. 8C).

### DETAILED DESCRIPTION OF THE INVENTION

The present invention relates to methods for promoting motor neuron survival, treating or preventing neurodegenerative disorders (e.g., amyotrophic lateral sclerosis (ALS)), identifying agents that promote survival of motor neurons, identifying agents that are useful for treating neurodegenerative disorders, diagnosing neurodegenerative disorders, predicting the progression of a neurodegenerative disorder in a subject, and monitoring the effectiveness of a therapy in reducing the progression of a neurodegenerative disorder in a subject.

The work described herein, inter alia, demonstrates that Aurora kinase inhibition can promote motor neuron survival. Surprisingly, and unexpectedly, the inventors have demonstrated that Aurora kinase inhibition increases survival of motor neurons in a concentration dependent manner.

Accordingly, in an aspect, disclosed herein is a method of promoting motor neuron survival, comprising contacting a motor neuron or a population of cells comprising a motor neuron with an effective amount of an agent that inhibits Aurora kinase. Aurora kinases are serine/threonine kinases involved in cell proliferation. In particular, Aurora kinases control chromatid segregation. To date, at least three mammalian Aurora kinases have been identified (e.g., Aurora A, Aurora B and Aurora C, also referred to as Aurora kinase A (AURKA; Gene ID: 6790), Aurora kinase B (AURKB; Gene ID: 9212), and Aurora Kinase C (AURKC; Gene ID: 6795), respectively). Aurora kinases in humans possess a N-terminal domain of 39 to 129 residues in length, a protein kinase domain, and a short C-terminal domain containing 15 to 20 residues. Aurora A acts during prophase of mitosis and is essential for proper centrosome functioning. Aurora B acts to attach mitotic spindle to the centromere. Aurora C reportedly acts in germ-line cells, but not much is known about its function.

As used herein, the phrase “promoting motor neuron survival” refers to an increase in survival of motor neuron cells as compared to a control. In some embodiments, contacting of a motor neuron with an agent described herein results in at least about 10%, 20%, 30%, 40%, 50% 60%, 70%, 80%, 90%, 95%, 100%, 2-fold, 3-fold, 4-fold, 5-fold or more increase in motor neuron survival relative to non-treated control.

Motor neuron survival can be assessed by for example (i) increased survival time of motor neurons in culture; (ii) increased production of a neuron-associated molecule in culture or in vivo, e.g., choline acetyltransferase, acetylcholinesterase, SMN or GEMs; or (iii) decreased symptoms of motor neuron dysfunction in vivo. Such effects may be measured by any method known in the art. In one non-limiting example, increased survival of motor neurons may be measured by the method set forth in Arakawa et al. (1990, J. Neurosci. 10:3507-3515); increased production of neuron-associated molecules may be measured by bioassay, enzymatic assay, antibody binding, Northern blot assay, etc., depending on the molecule to be measured; and motor neuron dysfunction may be measured by assessing the physical manifestation of motor neuron disorder. In one embodiment, the increase in motor neuron survival can be assessed by measuring the increase in SMN protein levels and/or GEM numbers. Cell survival can also be measured by uptake of calcein AM, an analog of the viable dye, fluorescein diacetate. Calcein is taken up by viable cells and cleaved intracellularly to fluorescent salts which are retained by intact membranes of viable cells. Microscopic counts of viable neurons correlate directly with relative fluorescence values obtained with the fluorometric viability assay. This method thus provides a reliable and quantitative measurement of cell survival in the total cell population of a given culture (Bozyczko-Coyne et al., J. Neur. Meth. 50:205-216, 1993). Other methods of assessing cell survival are described in U.S. Pat. Nos.: 5,972,639; 6,077,684 and 6417,160, contents of which are incorporated herein by reference.

In vivo motor neuron survival can be assessed by an increase in motor neuron, neuromotor or neuromuscular function in a subject. In one non-limiting example, motor neuron survival in a subject can be assessed by reversion, alleviation, amelioration, inhibition, slowing down or stopping of the progression, aggravation or severity of a condition associated with motor neuron dysfunction or death in a subject, e.g., ALS.

As used herein, “agent that inhibits Aurora kinase” refers to an agent that decreases the level of Aurora kinase mRNA or protein, an activity of Aurora kinase, the half-life of Aurora kinase mRNA or protein, or the binding of Aurora kinase to another molecule (e.g., a substrate for a Aurora kinase, see e.g., Kollareddy et al., “Aurora Kinases: Structure, Functions and their Association with Cancer,” 2008; 152(1):27-33). For example, the agent may directly or indirectly inhibit the ability of Aurora kinases to activate apoptotic pathways. Expression levels of mRNA can be determined using standard RNase protection assays or in situ hybridization assays, and the level of protein can be determined using standard Western or immunohistochemistry analysis. The phosphorylation level of a protein can also be measured using standard assays. In some embodiments, an agent that inhibits Aurora kinase decreases Aurora kinase activity by at least 20, 40, 60, 80, or 90%. In some embodiments, the level of Aurora kinase is at least 2, 3, 5, 10, 20, or 50-fold lower in the presence of the agent that inhibits Aurora kinase.

In some embodiments, the agent inhibits Aurora kinase (e.g., an Aurora kinase inhibitor). An Aurora kinase inhibitor or agent that inhibits Aurora kinase can be small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

The present invention contemplates any agent that inhibits a member of the Aurora kinase family. In some embodiments, the agent is a pan Aurora kinase inhibitor. In some embodiments, the agent is a dual inhibitor (e.g., of Aurora kinase A and Aurora kinase B). In some embodiments, the agent inhibits Aurora kinase A. In some embodiments, the agent inhibits Aurora kinase B. In some embodiments, the agent inhibits Aurora kinase C.

Exemplary agents that inhibit Aurora kinase include, but are not limited to, VX-608 (aka MK-0457, Tozasertib), ZM447439 (also known as N-[4-[[6-Methoxy-7-[3-(4-morpholinyl)propoxy]-4-quinazolinyl] amino]phenyl]benzamide), N-[4-[(6,7-Dimethoxy-4 quinazolinyl)amino] phenyl]benzamide hydrochloride (referred to herein as “4-4-Ben”), MLN8054, PHA-680632, TAK-901, AMG900, PF-03814735, CCT129202, phtalazinonepyrazole, hesperidin hydrochloride, CCT 137690, TC-A 2317 hydrochloride, Aurora kinase inhibitor II, Aurora Kinase Inhibitor III (also known as Cyclopropanecarboxylic acid {3-[4-(3-trifluoromethyl-phenylamino)-pyrimidin-2-ylamino]-phenyl}-amide); JNJ-7706621, H-1152, PHA739358, OM137, SNS-314, AT9283, CYC-116, MLN8237, ENMD-2076, SBE 13 hydrochloride, analogs or derivatives thereof, and combinations thereof.

Exemplary analogs of VX-608 which may be useful as an agent that inhibits Aurora kinase include the compounds and pharmaceutically acceptable salts of formulas Ia and I disclosed in PCT International Application Publication No. WO2012/112674 (incorporated by reference herein in its entirety). The VX-608 analogs can be assayed for their ability to inhibit Aurora kinase and promote motor neuron survival according to the methods described herein. Those skilled in the art will appreciate that a variety of routine methods are available for determining other suitable VX-608 analogs and derivatives.

In some embodiments, an agent that inhibits Aurora kinase (e.g., Aurora kinase inhibitor described herein) inhibits/lowers the activity of Aurora kinase by at least 5%, 10%, 20%, 30%, 40%, 50%, 60%, 70%, 80%, 90%, or 95% relative to a control. While not required, an agent that inhibits Aurora kinase can completely inhibit the Aurora kinase activity relative to a control.

In some embodiments, the agent increases activation of the anti-apoptotic protein kinase A pathway. In some embodiments, an agent that inhibits Aurora kinase (e.g., Aurora kinase inhibitor described herein) increases the activation of the anti-apoptotic protein kinase A pathway by at least 5%, 10%, 20%, 30%, 40%, 50%, 60%, 70%, 80%, 90%, 95%, or at least 1, 2, 3, 5, 10, 20, 50, or 100-fold more in the presence of the agent that inhibits Aurora kinase relative to a control.

In some embodiments, the agent increases phosphorylation of a protein in the anti-apoptotic protein kinase A pathway. In some embodiments, the protein is Bc1-2-associated death promoter (BAD). In some embodiments, an agent that inhibits Aurora kinase (e.g., Aurora kinase inhibitor described herein) increases the phosphorylation of a protein in the anti-apoptotic protein kinase A pathway (e.g., BAD), by at least 1, 2, 3, 5, 10, 20, 50, or 100-fold more in the presence of the agent that inhibits Aurora kinase relative to a control.

### Motor Neurons

The methods described herein are generally applicable to any motor neurons. In some embodiments, the motor neurons include, for example, HB9 motor neuron, a G93A motor neuron, a HB9(WT-SOD1) motor neuron, a HUES3 derived motor neuron, and combinations thereof.

In some embodiments, the motor neurons comprise a mutation in a gene associated with a neurodegenerative disorder. One non limiting example of a gene associated with a neurodegenerative disorder is SMN1. Another non limiting example of a gene associated with a neurodegenerative disorder is SOD1. A variety of SOD1 mutant alleles are known to be associated with SMA and/or ALS, including without limitation, SOD1A4V, SOD1G85R, and SOD1G93A.

In some embodiments, methods of the invention employ cells that are not motor neurons, wherein the cells can comprise a mutation in a gene associated with a neurodegenerative disorder. In one non-limiting example, some methods the present invention employ fibroblasts comprising a mutation in a gene associated with a neurodegenerative disorder. In some embodiments, methods of the invention employ fibroblasts comprising a mutation in a SOD1 gene, such as, without limitation, SOD1A4V, SOD1G85R, and SOD1G93A.

As used herein, the term “SOD1” refers to either the gene encoding superoxide dismutase 1 or the enzyme encoded by this gene. The SOD1 gene or gene product is known by other names in the art including, but not limited to, ALS1, Cu/Zn superoxide dismutase, indophenoloxidase A, IPOA, and SODC_HUMAN. Those of ordinary skill in the art will be aware of other synonymous names that refer to the SOD1 gene or gene product. The SOD1 enzyme neutralizes supercharged oxygen molecules (called superoxide radicals), which can damage cells if their levels are not controlled. The human SOD1 gene maps to cytogenetic location 21q22.1. Certain mutations in SOD1 are associated with ALS in humans including, but not limited to, Ala4Val, Gly37Arg, G85R and Gly93Ala, and more than one hundred others. Those of ordinary skill in the art will be aware of these and other human mutations associated with ALS. Certain compositions and methods of the present invention comprise or employ cells comprising a SOD1 mutation.

“SOD 1 mutations” refer to mutations in the SOD1 gene (NC_000021.8; NT_011512.11; AC_000064.1; NW_927384.1; AC_000153.1; NW_001838706.1 NM_000454.4; NP_000445.1 and NCBI Entrez GenelD: 6647) including but are not limited to Ala4Val, Cys6Gly , Val7Glu, Leu8Val, Glyl0Val, Glyl2Arg, Val14Met, Gly16Ala, Asn19Ser, Phe20Cys, Glu21Lys, Gln22Leu, Gly37Arg, Leu38Arg, Gly41Ser, His43Arg, Phe45Cys, His46Arg, Val47Phe, His48Gln, Glu49Lys, Thr54Arg, Ser59Ile, Asn65Ser, Leu67Arg, Gly72Ser, Asp76 Val, His80Arg, Leu84Phe, Gly85Arg, Asn86Asp, Val87Ala, Ala89Val, Asp90Ala, Gly93Ala, Ala95Thr, Asp96Asn, Val97Met, Glu100Gly, Asp101Asn, Ile104Phe, Ser105Leu , Leu106Val, Gly108Val, Ile112Thr, Ile113Phe, Gly114Ala, Arg115Gly, Val118Leu, Ala140Gly, Ala145Gly, Asp124Val, Asp124Gly, Asp125His, Leu126Ser, Ser134Asn, Asn139His, Asn139Lys, Gly141Glu, Leu144Phe, Leu144Ser, Cys146Arg, Ala145Thr, Gly147Arg, Val148Gly, Val148Ile, Ile149Thr, Ile151Thr, and Ile151Ser. SOD1 is also known as ALS, SOD, ALS1, IPOA, homodimer SOD1. “SOD 1 mutation” databases can be found at Dr. Andrew C. R. Martin website at the University College of London (www.bioinfo.org.uk), the ALS/SOD1 consortium website (www.alsod.org) and the human gene mutation database (HGMD®) at the Institute of Medical Genetics at Cardiff, United Kingdom.

### Contacting of Motor Neurons

Motor neurons or populations of cells comprising motor neurons can be contacted with the agents described herein in a cell culture e.g., in vitro or ex vivo, or administrated to a subject, e.g., in vivo. In some embodiments of the invention, an agent described herein can be administrated to a subject to treat, prevent, and/or diagnose neurodegenerative disorders, including those described herein. In some embodiments, a compound and/or agent described herein can be administered to a subject to treat, prevent, and/or diagnose ALS. In some embodiments, a compound and/or agent described herein can be administered to a subject to treat, prevent, and/or diagnose SMA.

The term “contacting” or “contact” as used herein in connection with contacting a motor neuron cell includes subjecting the cell to an appropriate culture media which comprises the indicated compound and/or agent. Where the motor neuron is in vivo, “contacting” or “contact” includes administering the compound and/or agent in a pharmaceutical composition to a subject via an appropriate administration route such that the compound and/or agent contacts the motor neuron in vivo. Measurement of cell survival can be based on the number of viable cells after period of time has elapsed after contacting of cells with a compound or agent. For example, number of viable cells can be counted after about at least 5 minutes, 10 minutes, 20 minutes, 30 minutes, 40 minutes, 50 minutes, 1 hour, 2 hours, 4 hours, 6 hours, 12 hours, 24 hours, 2 days, 3 days or more and compared to number of viable cells in a non-treated control.

For in vitro methods, motor neurons can be obtained from different sources. For example, motor neurons can be obtained from a subject, or derived from non motor neuron cells from a subject. In some embodiments, motor neuron is a whole cell. In some embodiments, the subject is suffering from a neurodegenerative disorder. In some embodiments, the subject is at risk of developing a neurodegenerative disorder. In some embodiments, the subject is suspected of having a neurodegenerative disorder. In some embodiments, the subject is at risk of developing a disorder characterized by neuronal cell death. In some embodiments, the subject is suspected of suffering from a disorder characterized by neuronal cell death. In some embodiments, the subject is suffering from neuronal cell death. In some embodiments, the subject is suffering from SMA. In some embodiments, the subject is suffering from ALS. In some embodiments, the subject is suffering from multiple sclerosis. In some embodiments, the subject is suffering from Parkinson's disease. In some embodiments, the subject is suffering from Huntington's disease. In some embodiments, the subject is a carrier e.g., a symptom-free carrier. In some embodiments, motor neuron cells are derived from a subject's embryonic stem cells (ESCs). In some embodiments, the subject is human. In some embodiments, the subject is mouse. In some embodiments, mouse is a transgenic mouse. Methods of inducing motor neuron differentiation from embryonic stem cells are known in the art, for example as described in Di Giorgio et al., Nature Neuroscience (2007), published online 15 April 2007; doi:10.1038/nn1885 and Wichterle et al., Cell (2002) 110:385-397. In some instances induced pluripotent stem cells can be generated from a subject and then differentiated into motor neurons. One exemplary method of deriving motor neurons from a subject is described in Dimos, J.T., et al. Science (2008) 321, 1218-122 (Epub Jul. 31, 2008).

For in vivo methods, a therapeutically effective amount of an agent described herein can be administered to a subject. Methods of administering agents to a subject are known in the art and easily available to one of skill in the art.

As one of skill in the art is aware, promoting survival of motor neuron cells in a subject can lead to treatment, prevention or amelioration of a number of neurodegenerative disorders. By “neurodegenerative disorder” is meant any disease or disorder caused by or associated with the deterioration of cells or tissues of the nervous system. In some instances, the neurodegenerative disorder is characterized by neuronal cell death (e.g., motor neurons and/or sensory neurons). Exemplary neurodegenerative disorders are polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, and Tabes dorsalis.

Those skilled in the art will also appreciate that the agents described herein can be used for promoting neuronal cell survival (e.g., motor neurons and/or sensory neurons). Promoting neuronal cell survival in a subject can lead to treatment, prevention or amelioration of a number of disorders characterized by neuronal cell death. Examples of neuronal cell death-related disorders or conditions that can be treated or can be prevented include, but are not limited to various neurodegenerative disorders (e.g. Alzheimer's disease, Huntington's Disease, prion diseases, Parkinson's Disease, amyotrophic lateral sclerosis, ataxia telangiectasia, spinobulbar atrophy, age-related reduction in number or in function, macular degeneration, retinal degeneration, dominant optic atrophy and Leber's hereditary optic neuropathy), diseases and conditions induced under various conditions of ischemia and/or excitotoxicity (e.g. ischemic stroke, hemorrhagic stroke and ischemic optic neuropathy), diseases due to nervous system trauma (e.g. spinal cord injury or traumatic optic neuropathy, or brain injury associated with physiological trauma), diseases due to inflammation (e.g. optic neuritis or multiple sclerosis), diseases due to infection (e.g. meningitis and toxoplasmosis optic neuropathy), diseases and conditions induced by certain medications or irrigating solutions (e.g. optic neuropathy induced by ethambutol or methanol), and diseases due to other etiologies (e.g. glaucoma).

The motor neuron diseases (MND) are a group of neurodegenerative disorders that selectively affect motor neurons, the nerve cells that control voluntary muscle activity including speaking, walking, breathing, swallowing and general movement of the body. Skeletal muscles are innervated by a group of neurons (lower motor neurons) located in the ventral horns of the spinal cord which project out the ventral roots to the muscle cells. These nerve cells are themselves innervated by the corticospinal tract or upper motor neurons that project from the motor cortex of the brain. On macroscopic pathology, there is a degeneration of the ventral horns of the spinal cord, as well as atrophy of the ventral roots. In the brain, atrophy may be present in the frontal and temporal lobes. On microscopic examination, neurons may show spongiosis, the presence of astrocytes, and a number of inclusions including characteristic “skein-like” inclusions, bunina bodies, and vacuolisation. Motor neuron diseases are varied and destructive in their effect. They commonly have distinctive differences in their origin and causation, but a similar result in their outcome for the patient: severe muscle weakness. Amyotrophic lateral sclerosis (ALS), primary lateral sclerosis (PLS), progressive muscular atrophy (PMA), pseudobulbar palsy, progressive bulbar palsy, spinal muscular atrophy (SMA) and post-polio syndrome are all examples of MND. The major site of motor neuron degeneration classifies the disorders. As used herein, the phrase “motor neuron degeneration” or “degeneration of motor neuron” means a condition of deterioration of motor neurons, wherein the neurons die or change to a lower or less functionally-active form.

Common MNDs include amyotrophic lateral sclerosis, which affects both upper and lower motor neurons. Progressive bulbar palsy affects the lower motor neurons of the brain stem, causing slurred speech and difficulty chewing and swallowing. Individuals with these disorders almost always have abnormal signs in the arms and legs. Primary lateral sclerosis is a disease of the upper motor neurons, while progressive muscular atrophy affects only lower motor neurons in the spinal cord. Means for diagnosing MND are well known to those skilled in the art. Non limiting examples of symptoms are described below.

### Amyotrophic Lateral Sclerosis (ALS)

Amyotrophic lateral sclerosis (ALS), also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. However, there is a familial form of ALS in adults, which often results from mutation of the superoxide dismutase gene, or SOD1, located on chromosome 21. In addition, a rare juvenile-onset form of ALS is genetic. Most individuals with ALS die from respiratory failure, usually within 3 to 5 years from the onset of symptoms. However, about 10 percent of affected individuals survive for 10 or more years.

### Spinal Muscular Atrophy (SMA)

Spinal muscular atrophy (SMA) refers to a number of different disorders, all having in common a genetic cause and the manifestation of weakness due to loss of the motor neurons of the spinal cord and brainstem. Weakness and wasting of the skeletal muscles is caused by progressive degeneration of the anterior horn cells of the spinal cord. This weakness is often more severe in the legs than in the arms. SMA has various forms, with different ages of onset, patterns of inheritance, and severity and progression of symptoms. Some of the more common SMAs are described below.

Defects in SMN gene products are considered as the major cause of SMA and SMN protein levels correlate with survival of subject suffering from SMA. The most common form of SMA is caused by mutation of the SMN gene. The region of chromosome 5 that contains the SMN (survival motor neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing. Some children also develop scoliosis (curvature of the spine) or other skeletal abnormalities. Affected children never sit or stand and the vast majority usually die of respiratory failure before the age of 2.

Symptoms of SMA type II usually begin after the child is 6 months of age. Features may include inability to stand or walk, respiratory problems, hypotonia, decreased or absent tendon reflexes, and fasciculations. These children may learn to sit but do not stand. Life expectancy varies, and some individuals live into adolescence or later.

Symptoms of SMA type III (Kugelberg-Welander disease) appear between 2 and 17 years of age and include abnormal gait; difficulty running, climbing steps, or rising from a chair; and a fine tremor of the fingers. The lower extremities are most often affected. Complications include scoliosis and joint contractures—chronic shortening of muscles or tendons around joints, caused by abnormal muscle tone and weakness, which prevents the joints from moving freely.

Other forms of SMA include e.g., Hereditary Bulbo-Spinal SMA Kennedy's disease (X linked, Androgen receptor), SMA with Respiratory Distress (SMARD 1) (chromosome 11, IGHMBP2 gene), Distal SMA with upper limb predominance (chromosome 7, glycyl tRNA synthase), and X-Linked infantile SMA (gene UBE1).

Current treatment for SMA consists of prevention and management of the secondary effect of chronic motor unit loss. Some drugs under clinical investigation for the treatment of SMA include butyrates, Valproic acids, hydroxyurea and Riluzole.

Symptoms of Fazio-Londe disease appear between 1 and 12 years of age and may include facial weakness, dysphagia (difficulty swallowing), stridor (a high-pitched respiratory sound often associated with acute blockage of the larynx), difficulty speaking (dysarthria), and paralysis of the eye muscles. Most individuals with SMA type III die from breathing complications.

Kennedy disease, also known as progressive spinobulbar muscular atrophy, is an X-linked recessive disease. Daughters of individuals with Kennedy disease are carriers and have a 50 percent chance of having a son affected with the disease. Onset occurs between 15 and 60 years of age. Symptoms include weakness of the facial and tongue muscles, hand tremor, muscle cramps, dysphagia, dysarthria, and excessive development of male breasts and mammary glands. Weakness usually begins in the pelvis before spreading to the limbs. Some individuals develop noninsulin-dependent diabetes mellitus.

The course of the disorder varies but is generally slowly progressive. Individuals tend to remain ambulatory until late in the disease. The life expectancy for individuals with Kennedy disease is usually normal.

Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids.

Progressive bulbar palsy, also called progressive bulbar atrophy, involves the bulb-shaped brain stem-the region that controls lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Affected persons have outbursts of laughing or crying (called emotional lability). Individuals eventually become unable to eat or speak and are at increased risk of choking and aspiration pneumonia, which is caused by the passage of liquids and food through the vocal folds and into the lower airways and lungs. Stroke and myasthenia gravis each have certain symptoms that are similar to those of progressive bulbar palsy and must be ruled out prior to diagnosing this disorder. In about 25 percent of ALS cases early symptoms begin with bulbar involvement. Some 75 percent of individuals with classic ALS eventually show some bulbar involvement. Many clinicians believe that progressive bulbar palsy by itself, without evidence of abnormalities in the arms or legs, is extremely rare.

Pseudobulbar palsy, which shares many symptoms of progressive bulbar palsy, is characterized by upper motor neuron degeneration and progressive loss of the ability to speak, chew, and swallow. Progressive weakness in facial muscles leads to an expressionless face. Individuals may develop a gravelly voice and an increased gag reflex. The tongue may become immobile and unable to protrude from the mouth. Individuals may also experience emotional lability.

Primary lateral sclerosis (PLS) affects only upper motor neurons and is nearly twice as common in men as in women. Onset generally occurs after age 50. The cause of PLS is unknown. It occurs when specific nerve cells in the cerebral cortex (the thin layer of cells covering the brain which is responsible for most higher level mental functions) that control voluntary movement gradually degenerate, causing the muscles under their control to weaken. The syndrome—which scientists believe is only rarely hereditary—progresses gradually over years or decades, leading to stiffness and clumsiness of the affected muscles. The disorder usually affects the legs first, followed by the body trunk, arms and hands, and, finally, the bulbar muscles. Symptoms may include difficulty with balance, weakness and stiffness in the legs, clumsiness, spasticity in the legs which produces slowness and stiffness of movement, dragging of the feet (leading to an inability to walk), and facial involvement resulting in dysarthria (poorly articulated speech). Major differences between ALS and PLS (considered a variant of ALS) are the motor neurons involved and the rate of disease progression. PLS may be mistaken for spastic paraplegia, a hereditary disorder of the upper motor neurons that causes spasticity in the legs and usually starts in adolescence. Most neurologists follow the affected individual's clinical course for at least 3 years before making a diagnosis of PLS. The disorder is not fatal but may affect quality of life. PLS often develops into ALS.

Progressive muscular atrophy (PMA) is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy hand movements, fasciculations, and muscle cramps. The trunk muscles and respiration may become affected. Exposure to cold can worsen symptoms. The disease develops into ALS in many instances.

Post-polio syndrome (PPS) is a condition that can strike polio survivors decades after their recovery from poliomyelitis. PPS is believed to occur when injury, illness (such as degenerative joint disease), weight gain, or the aging process damages or kills spinal cord motor neurons that remained functional after the initial polio attack. Many scientists believe PPS is latent weakness among muscles previously affected by poliomyelitis and not a new MND. Symptoms include fatigue, slowly progressive muscle weakness, muscle atrophy, fasciculations, cold intolerance, and muscle and joint pain. These symptoms appear most often among muscle groups affected by the initial disease. Other symptoms include skeletal deformities such as scoliosis and difficulty breathing, swallowing, or sleeping. Symptoms are more frequent among older people and those individuals most severely affected by the earlier disease. Some individuals experience only minor symptoms, while others develop SMA and, rarely, what appears to be, but is not, a form of ALS. PPS is not usually life threatening. Doctors estimate the incidence of PPS at about 25 to 50 percent of survivors of paralytic poliomyelitis.

In some embodiments, neurodegenerative disorder can be SMA or ALS.

By “treatment, prevention or amelioration of neurodegenerative disorder” is meant delaying or preventing the onset of such a disorder (e.g. death of motor neurons), at reversing, alleviating, ameliorating, inhibiting, slowing down or stopping the progression, aggravation or deterioration the progression or severity of such a condition. In one embodiment, the symptom of a neurodegenerative disorder is alleviated by at least 20%, at least 30%, at least 40%, or at least 50%. In one embodiment, the symptom of a neurodegenerative disorder is alleviated by more than 50%. In one embodiment, the symptom of a neurodegenerative disorder is alleviated by 80%, 90%, or greater. Treatment also includes improvements in neuromuscular function. In some embodiments, neuromuscular function improves by at least about 10%, 20%, 30%, 40%, 50% or more.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments of the invention, the subject suffers from a neurodegenerative disorder.

In some embodiments, the methods described herein further comprise selecting a subject diagnosed with a neurodegenerative disorder. A subject suffering from a neurodegenerative disorder can be selected based on the symptoms presented. For example a subject suffering from SMA may show symptoms of hypotonia, diminished limb movements, lack of tendon reflexes, fasciculations, tremors, swallowing, feeding difficulties, impaired breathing, scoliosis or other skeletal abnormalities, inability to stand or walk, abnormal gait, difficulty running, difficulty climbing steps, difficulty rising from a chair, and/or fine tremor of the fingers.

In some embodiments, the methods described herein further comprise selecting a subject at risk of developing a neurodegenerative disorder. A subject at risk of developing a neurodegenerative disorder can be selected based on a genetic diagnostic test (e.g., for a mutation in a gene associated with a neurodegenerative disorder (e.g., a mutation in the SOD1 gene, or in an SMN gene)) or based on the symptoms presented. For example a subject suffering from SMA may show symptoms of hypotonia, diminished limb movements, lack of tendon reflexes, fasciculations, tremors, swallowing, feeding difficulties, impaired breathing, scoliosis or other skeletal abnormalities, inability to stand or walk, abnormal gait, difficulty running, difficulty climbing steps, difficulty rising from a chair, and/or fine tremor of the fingers.

In some embodiments, the methods described herein further comprise selecting a subject suspected of having a neurodegenerative disorder. A subject suspected of having a neurodegenerative disorder can be selected based on a genetic diagnostic test (e.g., for a mutation in a gene associated with a neurodegenerative disorder (e.g., a mutation in the SOD1 gene, or in an SMN gene)) or based on the symptoms presented or a combination thereof. For example a subject suffering from SMA may show symptoms of hypotonia, diminished limb movements, lack of tendon reflexes, fasciculations, tremors, swallowing, feeding difficulties, impaired breathing, scoliosis or other skeletal abnormalities, inability to stand or walk, abnormal gait, difficulty running, difficulty climbing steps, difficulty rising from a chair, and/or fine tremor of the fingers.

### Diagnostic Tests, Monitoring Disease Progression, and Efficacy Of Treatment

Certain aspects of the disclosure relate to diagnostic tests and methods of diagnosing neurodegenerative disorders and/or disorders characterized by neuronal cell death. Other aspects of the disclosure relate to methods for monitoring progression of a neurodegenerative disease in a subject, and methods of monitoring the effectiveness of a therapy in reducing the progression of a neurodegenerative disorder in a subject.

In an aspect, a method for diagnosing a neurodegenerative disorder in a subject, comprises: (a) obtaining a biological sample comprising neuronal cells from the subject; (b) conducting at least one assay on the neuronal cells in the biological sample to detect the level or activity of Aurora kinase in the neuronal cells; and (c) diagnosing the subject as having a neurodegenerative disorder if the level or activity of the Aurora kinase in the neuronal cells is increased relative to a level or activity of Aurora kinase in a control sample.

In an aspect, a method for diagnosing a disorder characterized by neuronal cell death in a subject, comprises: (a) obtaining a biological sample comprising neuronal cells from the subject; (b) conducting at least one assay on the neuronal cells in the biological sample to detect the level or activity of Aurora kinase in the neuronal cells; and (c) diagnosing the subject as having a neurodegenerative disorder if the level or activity of the Aurora kinase in the neuronal cells is increased relative to a level or activity of Aurora kinase in a control sample.

In an aspect, a method for predicting the progression of a neurodegenerative disorder in a subject, comprises: (a) obtaining a first biological sample comprising neuronal cells from a subject diagnosed as having a neurodegenerative disorder; (b) obtaining a second biological sample comprising neuronal cells from the subject at a time which is later than when the first biological sample was obtained; (c) conducting at least one assay on the neuronal cells in the biological samples to detect a level or activity of Aurora kinase in the neuronal cells; and (d) predicting the progression of the neurodegenerative disorder in the subject, wherein: (i) the neurodegenerative disorder is predicted to progress if the level or activity of Aurora kinase in the neuronal cells in the second biological sample is increased relative to the level or activity of Aurora kinase in in the first biological sample; or (ii) the neurodegenerative disorder is not predicted to progress if the level or activity of Aurora kinase in the neuronal cells in the second biological sample is decreased relative to the level or activity of Aurora kinase in in the first biological sample.

In an aspect, a method of monitoring the effectiveness of a therapy in reducing the progression of a neurodegenerative disorder in a subject, comprises: (a) conducting at least one assay to determine the level or activity of Aurora kinase in a biological sample comprising neuronal cells from a subject having a neurodegenerative disorder prior to and following administration of the therapy to the subject; and (b) comparing the level or activity of Aurora kinase in the biological sample from the subject prior to the administration of the therapy to the level or activity of Aurora kinase in the biological sample from the subject following administration of the therapy; and (c) monitoring the effectiveness of the therapy in reducing the progression of the neurodegenerative disorder in the subject, wherein a decrease in the level or activity of Aurora kinase in the biological sample following administration of the therapy as compared to the level or activity of Aurora kinase in the biological sample prior to the administration of the therapy is an indication that the therapy is effective in reducing the progression of the neurodegenerative disorder in the subject. In some embodiments, an increase in the level or activity of Aurora kinase in the biological sample from the subject following administration of the therapy as compared to the level or activity of Aurora kinase in the biological sample prior to the administration of the therapy is an indication that the therapy is not effective in reducing the progression of the neurodegenerative disorder in the subject. In some embodiments, the absence of a change in the level or activity of Aurora kinase in the biological sample from the subject following administration of the therapy as compared to the level or activity of Aurora kinase in the biological sample prior to the administration of the therapy is an indication that the therapy is not effective in reducing the progression of the neurodegenerative disorder in the subject.

Any suitable control can be used. In some embodiments, the control is a subject that does not have the neurodegenerative disorder. In some embodiments, the control is a reference standard or level indicative of a subject that does not have the neurodegenerative disorder. In some embodiments, the control is a reference standard or level indicative of a subject for which progression of the neurodegenerative disorder is halted or reversed.

The disclosure contemplates using any assay that is capable of detecting the level or activity of Aurora kinase (e.g., Aurora A, Aurora B, Aurora C, etc.) in a cell.

In some embodiments, the at least one assay comprises a protein kinase assay to detect the phosphorylation activity of Aurora kinase. A protein kinase assay can be used to detect, for example, autophosphorylation of Aurora kinase or phosphorylation of a protein that interacts with Aurora kinase. Those skilled in the art will appreciate how to conduct kinase assays suitable for detecting the level or activity of Aurora kinase in a cell.

In some embodiments, the at least one assay comprises an assay that measures a level of Aurora kinase mRNA or protein in the neuronal cell. Any assays that are capable of measuring mRNA or protein in a cell can be used (e.g., hybridization assays (e.g., microarrays, qRT-PCR, etc.), sequencing assays (e.g., serial analysis of gene expression (SAGE), cap analysis of gene expression (CAGE), massively parallel signature sequencing (MPSS), GRO-seq, and RNA-seq) and immunological based assays (e.g., Western blotting, immunohistochemistry, flow cytometry, etc.). It should be appreciated by the skilled artisan that increased levels of Aurora kinase mRNA and/or protein in a neuronal cell relative to a control neuronal cell obtained from a subject that does not have the neurodegenerative disorder or a reference standard or level is indicative that the subject has or is at risk for developing the neurodegenerative disorder and/or a disorder characterized by neuronal cell death.

In some embodiments, the at least one assay comprises a phosphorylation assay to detect phosphorylation of Aurora kinase. The phosphorylation assay can be used to detect phosphorylation at any site of Aurora kinase which is indicative of Aurora kinase activity or activation. In some embodiments, the at least one assay comprises a phosphorylation assay to detect phosphorylation of Aurora kinase at threonine 288 (T288). In some embodiments, the at least one assay comprises a phosphorylation assay to detect phosphorylation of Aurora kinase at threonine 331 (S331). In some embodiments, the at least one assay comprises a protein kinase assay or phosphorylation assay to detect the level of phosphorylation of Aurora kinase

In some embodiments, the at least one assay comprises a protein kinase assay to detect the level of phosphorylation of a protein in the anti-apoptotic protein kinase A pathway. In some embodiments, the at least one assay comprises a protein kinase assay to detect the level of phosphorylation of BAD protein.

In some embodiments, the methods further comprise selecting a subject suspected of having a neurodegenerative disorder. In some embodiments, the diagnostic methods further comprise selecting a subject suspected of having a disorder characterized by neuronal cell death.

In some embodiments, the neuronal cells comprise motor neurons. In some embodiments, the neuronal cells comprise sensory neurons. In some embodiments, the neurodegenerative disorder is a neurodegenerative disorder described herein. In some embodiments, the disorder characterized by neuronal cell death is such a disorder described herein. In some embodiments, the neurodegenerative disorder is ALS. In some embodiments, the Aurora kinase is Aurora kinase A. In some embodiments, the Aurora kinase is Aurora kinase B. In some embodiments, the Aurora kinase is Aurora kinase C. In some instances, the Aurora kinase is a combination of at least two or more of Aurora kinase A, Aurora kinase B, and Aurora kinase C. In some embodiments, the Aurora kinase is a combination of Aurora kinase A, Aurora kinase B, and Aurora kinase C.

Those skilled in the art will appreciate that the effectiveness of any therapy in reducing the progression of a neurodegenerative disorder in a subject can be monitored in accordance with the methods described herein.

In some embodiments, the therapy comprises an agent that inhibits Aurora kinase selected from a pan Aurora kinase inhibitor, an inhibitor of Aurora kinase A, an inhibitor of Aurora kinase B, and an inhibitor of Aurora kinase C.

### Screening Assays

The present disclosure contemplates various assays for identifying agents that can increase motor neuron survival, as well as agents that can be used for treating neurodegenerative disorders (e.g., ALS or SMA). For example, candidate agents for increasing motor neuron survival can be identified by determining the effect of a test agent on a motor neuron, for example, where the motor neuron is cultured under conditions which minimize survival of motor neurons, e.g., withdrawal of one or more trophic factors, and where a greater number of motor neurons in the presence of a test agent relative to a control indicate that the test agent can promote motor neuron survival.

In an aspect, a method of identifying a candidate agent that promotes motor neuron survival comprises (a) contacting a population of cells comprising motor neurons with a test agent, and (b) measuring (i) the level or activity of an Aurora kinase or (ii) activation of the anti-apoptotic protein kinase A pathway, in the presence of the test agent, and (c) identifying the candidate agent that promotes motor neuron survival, wherein the test agent is a candidate agent for promoting motor neuron survival if the test agent (i) decreases the level or activity of the Aurora kinase or (ii) increases activation of the anti-apoptotic protein kinase A pathway, in the presence of the test agent.

In an aspect, a method of identifying a candidate agent for treating or preventing a neurodegenerative disorder comprises (a) contacting a population of cells comprising motor neurons with a test agent, and (b) measuring (i) the level or activity of an Aurora kinase or (ii) activation of the anti-apoptotic protein kinase A pathway, in the presence of the test agent, and (c) identifying the candidate agent for treating or preventing a neurodegenerative disorder, wherein the test agent is a candidate agent for treating or preventing a neurodegenerative disorder if the test agent (i) decreases the level or activity of the Aurora kinase or (ii) increases activation of the anti-apoptotic protein kinase A pathway, in the presence of the test agent.

In some embodiments, the contacting is performed in the absence of trophic factors. As used herein a “trophic factor” is a molecule that directly or indirectly affests the survival or function of a trophic factor responsive cell. Exemplary trophic factors include Ciliary Neurotrophic Factor (CNTF), basic Fibroblast Growth Factor (bFGF), insulin and insulin-like growth factors (e.g., IGF-I, IGF-H, IGF-IH), inteferons, interleukins, cytokines, and the neurotrophins, including Nerve Growth Factor (NGF), Neurotrophin-3 (NT-3), Neurotrophin-4/5 (NT-4/5) and Brain Derived Neurotrophic Factor (BDNF). A “trophic factor-responsive cell” is a cell which includes a receptor to which a trophic factor can specifically bind; examples include neurons (e.g., motor neurons) and non-neuronal cells (e.g., monocytes and neoplastic cells).

In one non-limiting example of the assay, motor neurons are optionally allowed to grow for a period time and trophic factors removed to induce cell death. Period of cell growth can be optimized depending on the assay format, initial plating density of the cells. In some embodiments, a practitioner can obtain cells that are already planted in the appropriate vessel and allowed to grow for a period of time. In other embodiments, the practitioner plates the cell in the appropriate vessel and allow the cells to grow for a period time, e.g., at least one day, at least two days, at least three days, at least four days, at least five days, at least six days, at least seven days or more before withdrawal of at least one trophic factor. In one embodiment, cells are grown for four days before withdrawal of at least one trophic factor.

After withdrawal of at least one trophic factor, the test agent is contacted or incubated with the motor neurons (e.g., a culture or population of cells comprising motor neurons). After a sufficient period of time, motor neurons that have survived are counted and their number compared to a control. In some embodiments, the MN death is induced within the first week using trophic factor withdrawal that is independent of the genotype of the cells. This allows implementing the assay in a straightforward and efficient screening (HTS) platform.

Generally, most motor neurons can survive well after two days in the absence of trophic supply (BDNF, GDNF and CNTF). A large proportion of MNs dies after three days in absence of trophic factors, and almost all MNs are killed after four days of starvation. Accordingly, motor neurons are allowed to grow for 2, 3, 4, 5, 6, or 7 days after withdrawal of the trophic factors before counting the motor neurons that survived after trophic factor withdrawal. In one embodiment, cell counting is three days after trophic factor withdrawal. Trophic factors are also referred to as neurotrophic factors or growth factors in the art.

A control can be a sample that is that is not contacted with a test agent. A control can be a sample that is treated with a known promoter of motor neuron survival. This can serve as a positive control. A control can be a sample that is treated with a known inhibitor of motor neuron survival.

Some exemplary promoters of motor neuron survival include, but are not limited to, kenpaullone, alsterpaullone, cycloheximide (CHX), and derivatives thereof. Additional promoters of motor neuron survival include those described, for example, in PCT/US2009/061468, filed Oct. 21, 2009, content of which is incorporated herein by reference in its entirety.

As used herein, the term “test agent” refers to agents and/or compositions that are to be screened for their ability to stimulate and/or increase and/or promote motor neuron survival. The test agents can include a wide variety of different compounds, including chemical compounds and mixtures of chemical compounds, e.g., small organic or inorganic molecules; saccharides; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the test agent is a small molecule.

The number of possible test agents runs into millions. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule, Pharmacopia, graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Depending upon the particular embodiment being practiced, the test agents can be provided free in solution, or may be attached to a carrier, or a solid support, e.g., beads. A number of suitable solid supports may be employed for immobilization of the test agents. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test agents may be screened individually, or in groups. Group screening is particularly useful where hit rates for effective test agents are expected to be low such that one would not expect more than one positive result for a given group.

Without limitations, motor neurons can be plated at any density that provides a optimal signal-to-noise ratio. For example, motor neurons can be plated at a density of 1,000 to 20,000 cells/well in a 384-well plate. In some embodiments, motor neurons are plated at density of 1,000; 2,000; 4,000; 8,000; 12,000; 16,000; or 20,000 cells/well in a 384-well plate. In one embodiment, motor neurons are plated at a density of 8,000 cells/well in a 384-well plate. Based on the foregoing, one of ordinary skill can adjust the plating density for other cell culturing vessels. For example one can calculate the dimensions of a well in the 384-well plate and the vessels to be used and scale the number of cells to be plated based on volume or surface area ratio between a well from the 384-well plate and the vessel to be used.

In some embodiments, the step of assessing motor neuron survival comprises detecting a motor neuron marker and a cell-replication marker. A selected test agent can be further limited to the agent where the motor neuron marker and the cell-replication marker co-localize in the same cell.

Any available method for identifying and counting motor neurons in a culture can be employed. For example, a motor neuron can comprise a detectable label for identification or counting. As used herein, the term “detectable label” refers to a molecule or an element or functional group in a molecule that allows for the detection, imaging, and/or monitoring of the presence the molecule. Without limitations, a detectable label can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 34-carboxy-pentyl)-3′ethyl-5,5′-dimethyloxacarbocyanine (CyA); 1H,5H,11H,15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17octahydro-inner salt (TR or Texas Red); BODIPY™ dyes; benzoxadiazoles; stilbenes; pyrenes; and the like. Many suitable forms of these fluorescent compounds are available and can be used.

In some embodiments, the motor neurons express a fluorescent protein. Examples of fluorescent proteins suitable for use as detectable label include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, morange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

A non-limiting list of fluorescent proteins incudes AceGFP, AcGFP1, AmCyan1, AQ143, AsRed2, Azami-Green (mAG), Cerulean, Cerulean, Citrine, cOFP, CopGFP, Cyan, CyPet, Dronpa, DsRed/DsRed2/DsRed-Express, DsRed-Monomer, EBFP, ECFP, EGFP, Emerald, eqFP611, EYFP, GFPs, HcRedl, HcRed-tandem, J-Red, Kaede, KFP, KikGR, mBanana, mCFP, mCherry, mCitrine, mEosEP, mHoneydew, MiCy, mKO, mOrange, mPlum, mRaspberry, mRFP1, mStrawberry, mTangerine, mYFP, mYFP, mYFP, PA-GFP, PA-mRFP, PhiYFP, PS-CFP-2, Renilla, tdFosFP, tdTomato, T-Sapphire, TurboGFP, UV-T-Sapphire, Venus, YPet, ZsYellowl, and derivatives and analogs thereof. In one embodiment, the fluorescent protein is Green Fluorescent Protein (GFP).

Specific devices or methods known in the art for the detection of fluorescence, e.g., from fluorophores or fluorescent proteins, include, but are not limited to, in vivo near-infrared fluorescence (see, e.g., Frangioni, Curr. Opin. Chem. Biol, 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, IEEE Transactions on Biomedical Engineering, 48:1034-1041 (2001), and the like. Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

A fluorescent protein can be expressed from a transgenic reporter gene in the motor neuron. Expression of the fluorescent protein from the transgenic reporter gene can be operably linked to expression of a motor neuron specific gene or can be under the control of a motor neuron specific promoter. Accordingly, in some embodiments, the sequence encoding the fluorescent protein is operably linked to a promoter element for a gene specific for motor neurons. In one embodiment, the sequence encoding the fluorescent protein is operably linked to one or more promoter elements from HB9 gene. Thus, in one embodiment, the motor neuron comprises a transgenic reporter gene comprising a fluorescent protein operably linked to one or more promoter elements from the HB9 gene.

In some embodiments, motor neurons are counted by an image-based method. Presence of a detectable label makes image-based method more amenable to automation. When the motor neurons express a fluorescent protein, surviving motor neurons can be those that are expressing the fluorescent protein when the counting is performed. In some embodiments, the number of motor neuron surviving after incubation with the test agent is at least about at least about 5%, 10%, 15%, 20%,25%, 30%, 35%, 40%, 45%,50%, 55%, 60%, 65%, 70%, 75%, 80%, 85%, 90%, 95%, 100%, 1.1-fold, 1.25-fold, 1.5-fold, 1.75-fold, 2-fold, 3-fold, 4-fold, 5-fold, 10-fold or higher than a control.

In some embodiments, number of motor neurons in a sample is assessed via automated image acquisition and analysis using a Cellomics ArrayScan VTI. The acquisition thresholds/parameters are established such that the computer-based calls of number of motor neurons are consistent with human-based calls. Such automated image acquisition and analysis allows for high-throughput screening of compounds.

Number of motor neurons can be assessed by: (i) increased total number of cells in the culture, as compared to an untreated control; (ii) increased total number of cells expressing a a detectable label in the test culture, as compared to an untreated control; (iii) increased ratio of cells expressing a detectable label to the total number of cells in the culture, as compared to an untreated control; or (iv) a combination thereof.

In some embodiments of this and other aspects described herein, the motor neurons can be cultured in the presence of non-neuronal cells. Without wishing to be bound by a theory, culturing in the presence of non-neuronal cells can identify compounds that do not act directly on motor neurons. In some embodiments, non-neuronal cells include glial cells.

The assay can be performed any suitable container or apparatus available to one of skill in the art for cell culturing. For example, the assay can be performed in 24-, 96-, or 384-well plates. In one embodiment, the assay is performed in a 384-well plate.

Motor neurons for the aspects disclosed herein can be obtained from any source available to one of skill in the art. Additionally, the motor neuron can be of any origin. Accordingly, in some embodiments, the motor neuron is a mammalian motor neuron. In one embodiment, the motor neuron is a human motor neuron or a mouse motor neuron. In one embodiment, motor neuron is a mouse ES cell-derived motor neuron.

In some embodiments, the motor neuron is from a subject, e.g., a patient. In some embodiments, the subject, e.g., a patient, is suffering from a neurodegenerative disorder. In some embodiments, the neurodegenerative disorder is ALS or SMA. In an embodiment the motor neuron is from a carrier, e.g., a symptom-free carrier.

In some embodiments, the screening method is a high-throughput screening. High-throughput screening (HTS) is a method for scientific experimentation that uses robotics, data processing and control software, liquid handling devices, and sensitive detectors. High-Throughput Screening or HTS allows a researcher to quickly conduct millions of biochemical, genetic or pharmacological tests. High-Throughput Screening are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen of an assay against a library of candidate compounds. An assay is a test for specific activity: usually inhibition or stimulation of a biochemical or biological mechanism. Typical HTS screening libraries or “decks” can contain from 100,000 to more than 2,000,000 compounds.

The key labware or testing vessel of HTS is the microtiter plate: a small container, usually disposable and made of plastic that features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, the researcher fills each well of the plate with the appropriate reagents that he or she wishes to conduct the experiment with, such as a motor neuron cell population. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

In another aspect, the invention provides a compound or agent selected by the screening assay described herein. It is to be understood that analogs, derivatives, isomers, and pharmaceutically acceptable salts of the compounds selected by the screening assays described herein are also claimed herein.

In yet another aspect, disclosed herein is a method for identifying a biological pathway that regulates or promotes motor neuron survival, the method comprising identifying a compound that promotes motor neuron survival using a method described herein, and establishing the cellular target of the compound, thereby determining whether the biological pathway comprising the cellular target regulates or promotes motor neuron survival.

In some embodiments, the test agent has known biological activity and/or cellular target(s). In some embodiments, the test agent is known to modulate a biological pathway.

### Methods of Treatment

Certain aspects of the present invention relate to methods for treating neurodegenerative disorders, and disorders characterized by neuronal cell death (e.g., motor neurons).

In an aspect, a method of treating or preventing a neurodegenerative disorder in a subject in need thereof comprises administering to the subject an effective amount of an agent that inhibits Aurora kinase.

In an aspect, a method of treating or preventing a disorder characterized by neuronal cell death in a subject in need thereof comprises administering to the subject an effective amount of an agent that inhibits Aurora kinase.

In some embodiments, the agent inhibits Aurora kinase and promotes motor neuron survival in the subject. In some embodiments, the agent inhibits Aurora kinase and ameliorates at least one symptom associated with the neurodegenerative disorder in the subject. In some embodiments, the agent inhibits Aurora kinase and treats the subject's neurodegenerative disorder. In some embodiments, the agent inhibits Aurora kinase and prevents the subject from developing a neurodegenerative disorder. In some embodiments, the agent inhibits Aurora kinase and prevents the subject's neurodegenerative disorder from progressing.

In some embodiments, the agent increases activation of the anti-apoptotic protein kinase A pathway and promotes motor neuron survival in the subject. In some embodiments, the agent increases activation of the anti-apoptotic protein kinase A pathway and ameliorates at least one symptom associated with the neurodegenerative disorder in the subject. In some embodiments, the agent increases activation of the anti-apoptotic protein kinase A pathway and treats the subject's neurodegenerative disorder. In some embodiments, the agent increases activation of the anti-apoptotic protein kinase A pathway and prevents the subject from developing a neurodegenerative disorder. In some embodiments, the agent increases activation of the anti-apoptotic protein kinase A pathway and prevents the subject's neurodegenerative disorder from progressing.

In some embodiments, the agent increases phosphorylation of a protein in the anti-apoptotic protein kinase A pathway and promotes motor neuron survival in the subject. In some embodiments, the agent increases phosphorylation of a protein in the anti-apoptotic protein kinase A pathway and ameliorates at least one symptom associated with the neurodegenerative disorder in the subject. In some embodiments, the agent increases phosphorylation of a protein in the anti-apoptotic protein kinase A pathway and treats the subject's neurodegenerative disorder. In some embodiments, the agent increases phosphorylation of a protein in the anti-apoptotic protein kinase A pathway and prevents the subject from developing a neurodegenerative disorder. In some embodiments, the agent increases phosphorylation of a protein in the anti-apoptotic protein kinase A pathway and prevents the subject's neurodegenerative disorder from progressing. In some embodiments, the protein in the anti-apoptotic protein kinase A pathway comprise Bc1-2-associated death promoter (BAD).

Any agent that inhibits Aurora kinase can be used. In some embodiments, the agent is a pan Aurora kinase inhibitor. In some embodiments, the agent inhibits Aurora kinase A. In some embodiments, the agent inhibits Aurora kinase B. In some embodiments, the agent inhibits Aurora kinase C. In some embodiments, the agent inhibits any combination of at least two of Aurora kinase A, Aurora kinase B, and Aurora kinase C. In some embodiments, the agent is selected from the group consisting of VX-608, ZM447439, 4-4-Ben, MLN8054, PHA-680632, TAK-901, AMG900, PF-03814735, CCT129202, phtalazinonepyrazole, hesperadin hydrochloride, CCT 137690, TC-A 2317 hydrochloride, Aurora kinase inhibitor II, JNJ-7706621, H-1152, PHA739358, OM137, SNS-314, AT9283, CYC-116, MLN8237, ENMD-2076, SBE 13 hydrochloride, analogs or derivatives thereof, and combinations thereof.

In some embodiments, the subject is a human.

In some embodiments, the subject selected for treatment of a neurodegenerative disorder or disorder characterized by neuronal cell death. In some embodiments, the subject is at risk of developing a neurodegenerative disorder or a disorder characterized by neuronal cell death. In some embodiments, the subject is suspected of having a neurodegenerative disorder or a disorder characterized by neuronal cell death. In some embodiments, the subject is suffering from a neurodegenerative disorder. The neurodegenerative disorder can be any neurodegenerative disorder described herein. In some embodiments, the neurodegenerative disorder is marked by neuronal cell death. In some embodiments, the neurodegenerative disorder is a motor neuron disease. In some embodiments, the neurodegenerative disorder is characterized by mutation of a SOD gene. In some embodiments, the neurodegenerative disorder is characterized by decreased levels of SOD protein. In some embodiments, the neurodegenerative disorder is ALS. In some embodiments, the neurodegenerative disorder is SMA.

In some embodiments, another therapeutic agent is also administered to the subject. Such a therapeutic agent can be administered in the same formulation or in separate formulations Ex e.g., Butyrates, Valproic acid, Hydroxyurea or Riluzole. In some embodiments, the agents described herein are used in combination with another therapeutic agent suitable for use in treating one or more symptoms of ALS, including, but not limited to, one or more of (i) hydrogenated pyrido [4,3-b] indoles or pharmaceutically acceptable salts thereof and (ii) agents that promote or increase the supply of energy to muscle cells, COX-2 inhibitors, poly(ADP-ribose)polymerase-1 (PARP-I) inhibitors, 3OS ribosomal protein inhibitors, NMDA antagonists, NMDA receptor antagonists, sodium channel blockers, glutamate release inhibitors, K(V)4.3 channel blockers, anti-inflammatory agents, 5-HT1A receptor agonists, neurotrophic factor enhancers, agents that promote motoneuron phenotypic survival and/or neuritogenesis, agents that protect the blood brain barrier from disruption, inhibitors of the production or activity of one or more proinflammatory cytokines, immunomodulators, neuroprotectants, modulators of the function of astrocytes, antioxidants (such as small molecule catalytic antioxidants), free radical scavengers, agents that decrease the amount of one or more reactive oxygen species, agents that inhibit the decrease of non-protein thiol content, stimulators of a normal cellular protein repair pathway (such as agents that activate molecular chaperones), neurotrophic agents, inhibitors of nerve cell death, stimulators of neurite growth, agents that prevent the death of nerve cells and/or promote regeneration of damaged brain tissue, cytokine modulators, agents that reduce the level of activation of microglial cells, cannabinoid CB1 receptor ligands, nonsteroidal anti-inflammatory drugs, cannabinoid CB2 receptor ligands, creatine, creatine derivatives, stereoisomers of a dopamine receptor agonist such as pramipexole hydrochloride, ciliary neurotrophic factors, agents that encode a ciliary neurotrophic factor, glial derived neurotrophic factors, agents that encode a glial derived neurotrophic factor, neurotrophin 3, agents that encode neurotrophin 3, or any combination thereof.

In some embodiments, the agents described herein are used in combination with another therapeutic agent suitable for use in treating one or more symptoms of SMA, including, but not limited to, one or more of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Estraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate; Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Morniflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolie Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium), or anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine),In some embodiments, the method further comprises identifying a biological pathway that regulates the SMN level in a cell using a method described herein.

In one aspect, the invention features a kit comprising an agent identified by the method described herein, and instructions to treat a neurodegenerative disorder e.g. ALS or SMA using a method described herein.

### Formulations and Administration

For administration to a subject, the agents described herein can be administered orally, parenterally, for example, subcutaneously, intravenously, intramuscularly, intraperitoneally, by intranasal instillation, or by application to mucous membranes, such as, that of the nose, throat, and bronchial tubes. One method for targeting the nervous system, such as spinal cord glia, is by intrathecal delivery. The targeted agent is released into the surrounding CSF and/or tissues and the released compound can penetrate into the spinal cord parenchyma, just after acute intrathecal injections. For a comprehensive review on drug delivery strategies including CNS delivery, see Ho et al., Curr. Opin. Mol. Ther. (1999), 1:336-3443; Groothuis et al., J. Neuro Virol. (1997), 3:387-400; and Jan, Drug Delivery Systmes: Technologies and Commercial Opportunities, Decision Resources, 1998, content of all which is incorporate herein by reference.

They can be administered alone or with suitable pharmaceutical carriers, and can be in solid or liquid form such as, tablets, capsules, powders, solutions, suspensions, or emulsions.

As used herein, the term “administered” refers to the placement of an agent described herein, into a subject by a method or route which results in at least partial localization of the compound at a desired site. An agent described herein can be administered by any appropriate route which results in effective treatment in the subject, i.e. administration results in delivery to a desired location in the subject where at least a portion of the composition delivered. Exemplary modes of administration include, but are not limited to, injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The agents can be formulated in pharmaceutically acceptable compositions which comprise a therapeutically-effective amount of the agent, formulated together with one or more pharmaceutically acceptable carriers (additives) and/or diluents. The agents can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds and/or agents can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 353,270,960.

As used here, the term “pharmaceutically acceptable” refers to those compounds, agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

The agents can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds and/or agents can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171. Methods to make invention formulations include the step of bringing into association or contacting an active agent with one or more excipients or carriers. In general, the formulations are prepared by uniformly and intimately bringing into association one or more agents with liquid excipients or finely divided solid excipients or both, and then, if appropriate, shaping the product.

The preparative procedure may include the sterilization of the pharmaceutical preparations. The agents may be mixed with auxiliary agents such as lubricants, preservatives, stabilizers, salts for influencing osmotic pressure, etc., which do not react deleteriously with the agents.

Examples of injectable form include solutions, suspensions and emulsions. Injectable forms also include sterile powders for extemporaneous preparation of injectible solutions, suspensions or emulsions. The agents of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. A suitable carrier is 5% dextrose in saline. Frequently, it is desirable to include additives in the carrier such as buffers and preservatives or other substances to enhance isotonicity and chemical stability.

In some embodiments, agents described herein can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

In one embodiment, the agents are prepared with carriers that will protect the compound and/or agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and PRIMOGEL™, and the like.

The agents can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds and/or agents may be incorporated with excipients and used in the form of tablets, capsules, elixirs, suspensions, syrups, and the like. Such compositions and preparations should contain at least 0.1% of compound and/or agent. The percentage of the agent in these compositions may, of course, be varied and may conveniently be between about 2% to about 60% of the weight of the unit. The amount of compound and/or agent in such therapeutically useful compositions is such that a suitable dosage will be obtained. Preferred compositions according to the present invention are prepared so that an oral dosage unit contains between about 100 and 2000 mg of compound and/or agent.

Examples of bases useful for the formulation of suppositories are oleaginous bases such as cacao butter, polyethylene glycol, lanolin, fatty acid triglycerides, witepsol (trademark, Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

The agents can also be administrated directly to the airways in the form of an aerosol. For administration by inhalation, the agents in solution or suspension can be delivered in the form of an aerosol spray from pressured container or dispenser which contains a suitable propellant, e.g., a gas such as carbon dioxide, or hydrocarbon propellant like propane, butane or isobutene. The agents can also be administrated in a no-pressurized form such as in an atomizer or nebulizer.

The agents can also be administered parenterally. Solutions or suspensions of these agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of agent calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the agents are formulated into ointments, salves, gels, or creams as generally known in the art.

The agents can be administrated to a subject in combination with other pharmaceutically active agents. Exemplary pharmaceutically active compounds and/or agents include, but are not limited to, those found in Harrison's Principles of Internal Medicine, 13^{th} Edition, Eds. T. R. Harrison et al. McGraw-Hill N.Y., NY; Physician's Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference. In some embodiments, the pharmaceutically active agent is selected from the group consisting of butyrates, valproic acid, hydroxyuirae and Riluzole.

The agents and the other pharmaceutically active agent can be administrated to the subject in the same pharmaceutical composition or in different pharmaceutical compositions (at the same time or at different times). For example, an Aurora kinase inhibitor and an additional agent for treating a neurodegenerative disorder can be administrated to the subject in the same pharmaceutical composition or in different pharmaceutical compositions (at the same time or at different times).

The amount of agent which can be combined with a carrier material to produce a single dosage form will generally be that amount of the agent which produces a therapeutic effect. Generally out of one hundred percent, this amount will range from about 0.1% to 99% of compound, preferably from about 5% to about 70%, most preferably from 10% to about 30%.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

The pharmaceutical compositions can be included in a container, pack, or dispenser together with instructions for administration.

As used herein, the term “therapeutically effective amount” means an amount of the compound and/or agent which is effective to promote the survival of motor neuron cells or to prevent or slow the death of such cells. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Guidance regarding the efficacy and dosage which will deliver a therapeutically effective amount of a compound and/or agent to treat ALS or SMA can be obtained from animal models of ALS or SMA, see e.g., those described in Hsieh-Li et al. Nature Genetics. 2000; 24:66-70 and references cited therein.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds and/or agents lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, GDF-8 binding assays, and immunological assays.

The dosage may be determined by a physician and adjusted, as necessary, to suit observed effects of the treatment. Generally, the compositions are administered so that the compound and/or agent is given at a dose from 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For antibody compounds and/or agents, one preferred dosage is 0.1 mg/kg of body weight (generally 10 mg/kg to 20 mg/kg). If the antibody is to act in the brain, a dosage of 50 mg/kg to 100 mg/kg is usually appropriate.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dos can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or more times daily.

### Kits

An agent described herein can be provided in a kit. The kit includes (a) the agent, e.g., a composition that includes the agent, and (b) informational material. The informational material can be descriptive, instructional, marketing or other material that relates to the methods described herein and/or the use of the agent for the methods described herein. For example, the informational material describes methods for administering the agent to promote motor neuron survival, treat or prevent a neurodegenerative disorder (e.g., ALS or SMA), or at least one symptom of disease neurodegenerative disorder, or a disease associated with dysfunctional or decreases motor neurons.

In one embodiment, the informational material can include instructions to administer the agent in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the agent, the composition of the kit can include other ingredients, such as a solvent or buffer, a stabilizer or a preservative, and/or a second agent for treating a condition or disorder described herein, e.g. increased pancreatic islet mass. Alternatively, the other ingredients can be included in the kit, but in different compositions or containers than the agent. In such embodiments, the kit can include instructions for admixing the agent and the other ingredients, or for using the agent together with the other ingredients.

The agent can be provided in any form, e.g., liquid, dried or lyophilized form. It is preferred that the agent be substantially pure and/or sterile. When the agent is provided in a liquid solution, the liquid solution preferably is an aqueous solution, with a sterile aqueous solution being preferred. When the compound and/or agent is provided as a dried form, reconstitution generally is by the addition of a suitable solvent. The solvent, e.g., sterile water or buffer, can optionally be provided in the kit.

The kit can include one or more containers for the composition containing the compound and/or agent. In some embodiments, the kit contains separate containers, dividers or compartments for the agent (e.g., in a composition) and informational material. For example, the agent (e.g., in a composition) can be contained in a bottle, vial, or syringe, and the informational material can be contained in a plastic sleeve or packet. In other embodiments, the separate elements of the kit are contained within a single, undivided container. For example, the agent (e.g., in a composition) is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent (e.g., in a composition). For example, the kit includes a plurality of syringes, ampules, foil packets, or blister packs, each containing a single unit dose of the agent. The containers of the kits can be air tight and/or waterproof.

The compound and/or agent (e.g., in a composition) can be administered to a subject, e.g., an adult subject, e.g., a subject in need of motor neurons. The method can include evaluating a subject, e.g., to evaluate motor neuron survival, and thereby identifying a subject as having decreased motor neurons or being pre-disposed to motor neuron death or dysfunction.

### Some Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the term “modulate” means to cause or facilitate a qualitative or quantitative change, alteration, or modification in a molecule, a process, pathway, or phenomenon of interest. Without limitation, such change may be an increase, decrease, a change in binding characteristics, or change in relative strength or activity of different components or branches of the process, pathway, or phenomenon.

The term “modulator” refers to any molecule or compound that causes or facilitates a qualitative or quantitative change, alteration, or modification in a process, pathway, or phenomenon of interest. As used herein, the term “modulator” comprises both inhibitors and activators of a biological pathway or target.

As used herein, the phrase “modulation of a biological pathway” refers to modulation of activity of at least one component of the biological pathway. It is contemplated herein that modulator of the signaling pathway can be, for example, a receptor ligand (e.g., a small molecule, an antibody, an siRNA), a ligand sequestrant (e.g., an antibody, a binding protein), a modulator of phosphorylation of a pathway component or a combination of such modulators.

One of skill in the art can easily test a compound to determine if it modulates a signaling pathway by assessing, for example, phosphorylation status of the receptor or expression of downstream proteins controlled by the pathway in cultured cells and comparing the results to cells not treated with a modulator. A modulator is determined to be a signaling pathway modulator if the level of phosphorylation of the receptor or expression of downstream proteins in a culture of cells is reduced by at least 20% compared to the level of phosphorylation of the receptor or expression of downstream proteins in cells that are cultured in the absence of the modulator; preferably the level of phosphorylation is altered by at least 30%, at least 40%, at least 50%, at least 60%, at least 70%, at least 80%, at least 90%, at least 95%, or at least 99% in the presence of a pathway modulator.

The terms “decrease” , “reduced”, “reduction” , “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

The terms “increased” ,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

As used herein, an “RNA interference molecule” refers to a compound which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. The succession of letters used as abbreviations for bases) that biochemically characterizes a specific nucleic acid. A polynucleotide sequence presented herein is presented in a 5′ to 3′ direction unless otherwise indicated.

The nucleic acid molecules that modulate the biological pathways or targets described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Identification of Aurora Kinase Inhibitors as ALS Therapeutics in a Small Molecule Screen on Stem-Cell-Derived Motor Neurons

Amyotrophic Lateral Sclerosis (ALS) is a late-onset neurodegenerative disease that affects both spinal cord and cortical motor neurons (MNs). The pathogenic processes underlying ALS are multifactorial and not fully determined at present. Although no genetic component is apparent in 90% of ALS cases, referred to as sporadic, the remaining 10% are familial, typically inherit the disease in an autosomal dominant manner (Cleveland DW and Rothstein JD., 2001; Bruijn L I., 2004; et al; Brown R H., 1997). Within the familial forms of ALS, approximately 20% are caused by mutations in the Cu/Zn SOD1 gene, and a further 3%-4% of familial cases are due to pathogenic variants in either the TAR DNA-binding protein 43 (TDP-43) or Fused in Sarcoma (FUS) gene (Rosen D R., 1993; Arai, T., et al 2006; Neumann, M., et al 2006; Kwiatkowski, T. J., Jr., et al 2009), although many other genes have been associated with familial ALS (Andersen, P. M. and A. Al-Chalabi., 2011). The very recent identification of a hexanucleotide repeat expansion within the C9orf72 gene points to it as potentially the most frequent pathogenic cause of ALS identified thus far, accounting overall for 6% of the sporadic ALS cases and between 30-40% of familial ALS cases in Europe and the USA (Renton, A. E., et al., 2011; Majounie, E., et al., 2012). Among various genes involved in familial ALS, SOD1 linked ALS is the best understood form of the disease due to the early discovery of disease-causing mutations and the availability of animal models. It is the toxicity of the mutant SOD1 protein, rather than the deficiency of the normal SOD1 protein, that is thought to lead to disease progression (Rosen D R et al., 1993). Although how exactly mutations in SOD1 gene cause MN death is still unclear, it is now well accepted that both cell-autonomous and non-cell-autonomous mechanisms contribute to MN degeneration in ALS (Clement A M et al., 2003; Boillee S et al., 2006; Haidet-Phillips A M et al., 2011; Philips, T. and W. Robberecht, 2011).

ALS is the most common MN disease in adults, yet there are no effective treatments for it. The sole approved drug, Riluzole, extends life by only a few months with very little functional improvement (Miller et al., 2007). Recently, two promising drug candidates olesoxime and dexpramipexole (Cudkowicz et al., 2011; Sunyach et al., 2012) failed in phase III clinical trials. Previously, we have carried out small molecule survival screen using MN generated from wild type mouse ESCs (Hb9::GFP) and from mouse ESCs carrying a human SOD1^{G93A} transgene (SOD1^{G93A}/Hb9::GFP) to identify those that can promote MN survival (Yang et al., 2013). Kenpaullone, a multikinase inhibitor, was identified as a hit that showed impressive ability to prolong the healthy survival of both types of MNs. Furthermore, Kenpaullone also was able to improve the survival of human MNs derived from wild-type and numerous ALS-patient-iPSCs, thus, indicating potential to be developed as an ALS therapeutic (Yang et al., 2013). Although Kenpaullone showed a robust cell survival effect but it is highly non-specific and promoted MN survival by inhibiting multiple kinases which could be a hindrance to therapeutic development. We have been screening several other highly specific kinase inhibitors to find one that can promote MN survival and demonstrate high target specificity. Among many kinase inhibitors we have screened, we have found several molecules of a class of cell cycle kinase inhibitors namely Aurora Kinase Inhibitors (AKIs) that remarkably promote both type of MN survival. To further validate the target specificity of the hits we tested numerous AKIs that are currently in cancer clinical trials and found that most of them effectively enhanced MN survival. We further demonstrate that AKIs not only maintain MN survival over a long period of time both in the absence and presence of trophic factors but also maintain neuronal processes and synapses. Furthermore, we show that growth factor withdrawal leads to Aurora kinase activation in degenerating MNs and that AKIs promote survival of MNs by activating the anti-apoptotic PKA pathway (Downward et al., 1999; Komaki et al., 2012; Lizcano et al., 2000). Lastly, we show that AKIs also improve survival of MNs produced from human ESCs, wild type and numerous patient derived iPSCs as well as preserve their morphology and ability to make synapses.

### Results

### Aurora Kinase Inhibitors Promote MN Survival

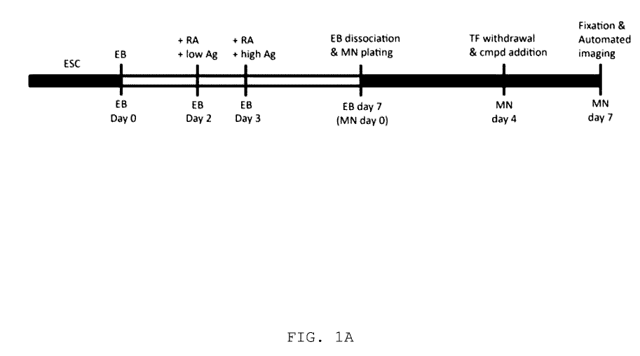

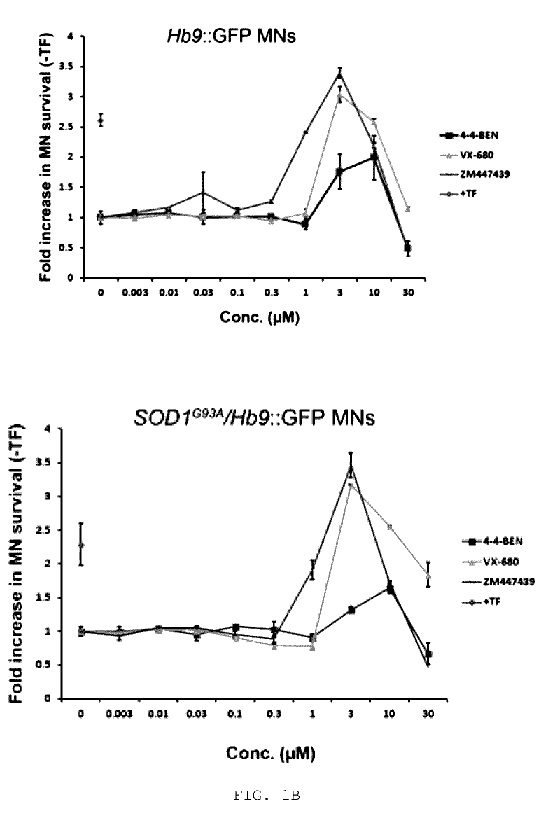

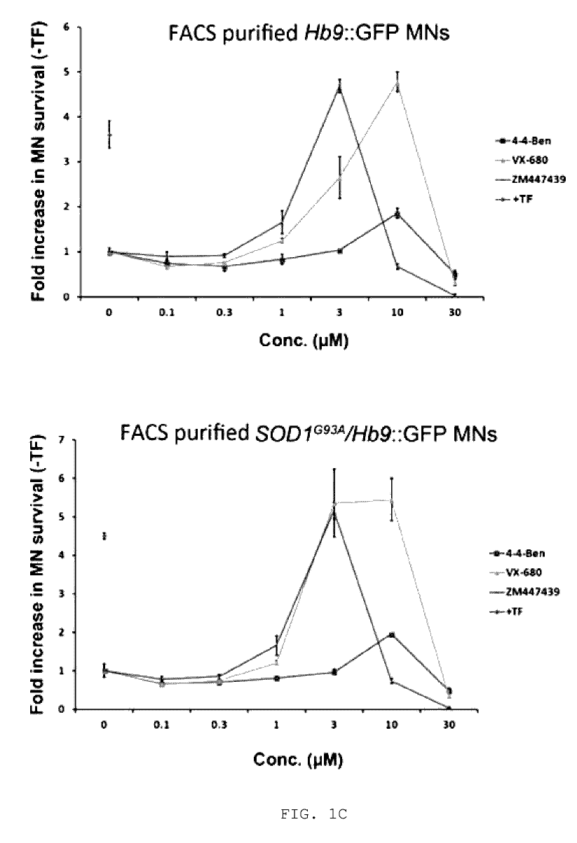

In the present study, we have screened highly specific small molecule kinase inhibitors to identify those which could promote MN survival. For the screen, we generated MNs from wild-type (Hb9::GFP) and SOD1^{G93A} (SOD1^{G93A}/Hb9::GFP) mouse ESCs using published protocols (Di Giorgio et al., 2007). We withdrew trophic factors as a standard method to induce MN death. Cell plating density as well as timing for initiation and duration of trophic factor withdrawal were used exactly as published to achieve ˜80% MN death in 3 days (FIG. 1A) (Yang et al., 2013). Compounds that strongly increased the number of surviving MNs and also preserved cell morphology were classified as hits. N-[4-[(6, 7-Dimethoxy-4-quinazolinyl) amino] phenyl] benzamide Hydrochloride (=4-4-Ben), VX-680 (MK-0457, Tozasertib) and ZM447439, annotated as AKIs, consistently increased the survival of MNs. We continued to work with this compound class as it reproducibly promoted survival of both MNs types and the effect was robust. We further reasoned that since this compound class promoted survival of both MN types, the probability of its working in other contexts such as on human MNs and in the ALS mouse model was maximum.

### Specific Aurora Kinase Inhibitors Promote MN Survival

First, we examined the survival effect of 4-4-Ben, VX-680 and ZM447439 in a dose dependent assay and found VX-680 and ZM447439 to be the most effective and potent in promoting MN survival (FIG. 1B). High expression of Aurora kinases is associated with various types of cancer (Sen et al., 1997; Reichardt et al., 2003; Tchatchou et al., 2007) and there are many AKIs including VX-680 and ZM447439 that are in clinical trials for anti-cancer drug development. We also tested several of these AKIs that are in clinical trials and found that they also effectively rescued MN survival. In follow-up studies, we focused on VX-680 and ZM447439 as they were the most potent and robust at increasing survival of both wild type and mutant MNs.

### The Effect of Aurora Kinase Inhibitors is Cell Autonomous

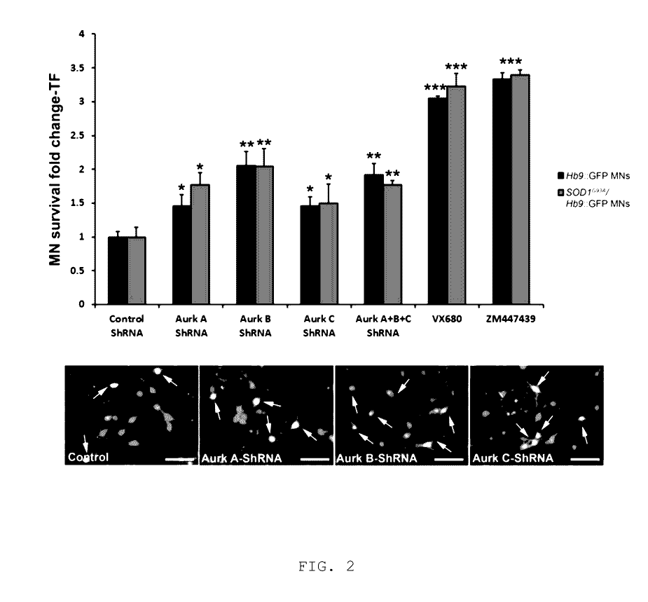

ESC-derived MN culture achieves ˜30%-50% MN differentiation efficiency and is therefore highly heterogeneous. To examine whether AKIs specifically act in a cell-autonomous or non-cell-autonomous manner, we established a highly enriched MN culture through FACS purification based on Hb9::GFP reporter expression. When trophic factors were withdrawn from purified MN cultures to induce death, AKIs (4-4-Ben, VX-680 & ZM447439) rescued MN survival in a dose dependent fashion similar to that of mixed culture, indicating that they work in a cell-autonomous manner (FIG. 1C). Next, we asked whether AKIs exert their survival promoting effect only cell-autonomously or whether they also function non-cell-autonomously to rescue MN death. To address this question we produced astrocyte conditioned medium (ACM) by incubating primary murine astrocytes with AKIs (VX-680 and ZM447439). The ACM was then dialyzed using a dialyzer with pore size appropriate for eliminating residual AKIs. Un-dialyzed ACM showed a dilution dependent survival promoting effect on both mixed and FACS purified MN cultures (Supp FIGS. 2A and 2B). Dialyzed ACM was unable to rescue MN death. These data demonstrate that AKIs exert their survival effect in a cell-autonomous manner.

### ShRNA Knock-Down of Aurora Kinase Validate the Target Specificity of AKIs

To further determine target specificity of the identified AKIs, we knocked down Aurora kinase expression in MNs. First, we examined the endogenous expression levels of Aurora kinases in MNs. There are three subtypes of Aurora kinases reported in mammalian cells known as a, b and c (Carmena et al., 2003). Expression of all three Aurora kinase subtypes was observed in mixed and FACS purified MN cultures by PCR as well as by western blot. We then generated lentiviruses expressing shRNAs for Aurora kinase a, b and c separately. Knockdown of all three subtypes significantly increased the survival of both Hb9::GFP and SOD1^{G93A} MNs, with type b showing the greatest effect (FIG. 2). Simultaneous knock-down of all the three subtypes did not produce a synergistic effect. The knockdown-mediated survival effect was less robust than that of either VX-680 or ZM447439 alone. This could be due to insufficient knock-down or additional targets of the compounds.

### Aurora Kinase is Activated in Dying MNs

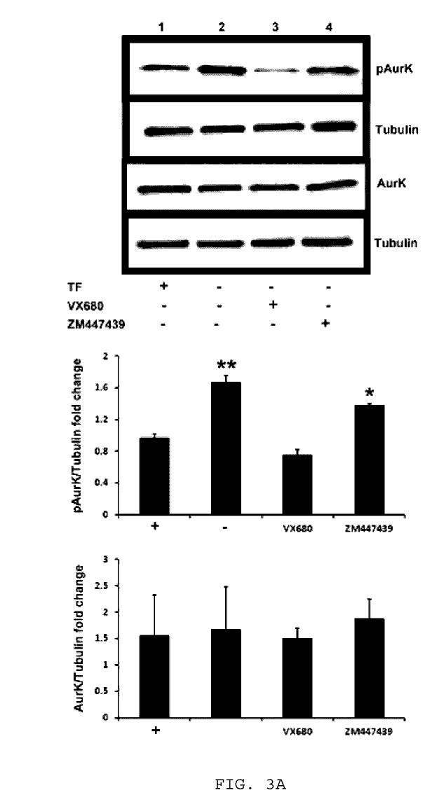

We reasoned that if AKIs exert their MN survival effect by inhibiting Aurora kinases, then Aurora kinases should be highly active in dying MNs compared to their stable counterparts. Aurora kinase is active when phosphorylated at threonine 288 (T288) or serine 331 (S331) (Eleni Petsalaki; JCB 2011, David L. Satinover; PNAS 2004). To examine Aurora kinase activation in dying MNs, we measured phospho-Aurora kinase expression in FACS purified Hb9::GFP cultures 48hrs after trophic factor withdrawal. As anticipated, phosphorylated Aurora kinase levels were increased significantly in the absence of trophic factors compared to cultures in which trophic factors were maintained (FIG. 3A). Importantly, AKIs blocked the increase in phosphorylated Aurora kinase levels initiated by trophic factors withdrawal. This could be due to self-phosphorylation of Aurora kinases (Eleni Petsalaki; JCB 2011, David L. Satinover; PNAS 2004) which is inhibited in the presence of AKIs. There was no change in level of total Aurora kinase (FIG. 3A). Increased levels of phospho Aurora kinase were also observed by immunostaining in Hb9::GFP MNs in the absence of trophic factors (FIG. 3B).

### AKIs Promote Long Term Survival of MNs

To investigate whether AKIs promote MN survival for an extended period of time, we choose to test ZM447439 as it was the most effective and potent of all AKIs assayed. Trophic factors were removed and ZM447439 was added to the cultures as previously and MN survival was analyzed on day 7, 14 & 21. ZM447439 increased survival of both Hb9::GFP and SOD1^{G93A}/Hb9::GFP MNs at all time points. We also found that the basal MN death that occurs even in presence of neurotrophic factors could also be prevented by ZM447439 treatment over the same time course.

### AKIs Preserve MN Morphology and Function

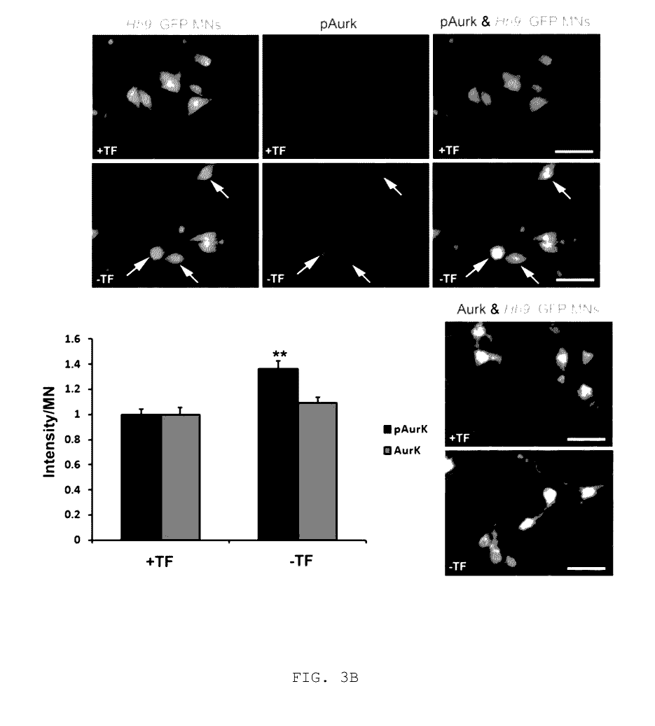

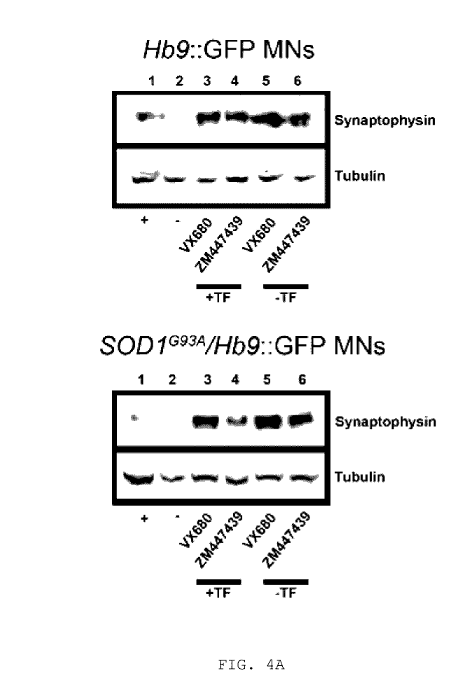

To examine whether MNs kept alive by AKIs maintain morphological integrity we carried out a series of analyses on surviving MNs in the absence of trophic factors. First, we analyzed MN morphology by comparing total neurite length, maximum neurite length, number of extremities and number of nodes per MN and found that ZM447439 preserved all of these features in surviving Hb9::GFP and SOD1^{G93A}/Hb9::GFP MNs even at extended time point. We also used an automated imager to analyze the number of synapses per MN by counting co-localized regions positive for the presynaptic marker synapsin and the postsynaptic marker PSD-95. Both VX-680 and ZM447439 preserved the number of synapses in Hb9::GFP and SOD1^{G93A}/Hb9::GFP MNs in the absence of trophic factors (FIG. 4B). Interestingly, VX-680 and ZM447439 increased the number of synapses per MN even when cells were kept in neurotrophin containing medium (FIG. 4B). We also found increased expression of the synaptic protein synaptophysin by immunoblotting in both Hb9::GFP and SOD1^{G93A}/Hb9::GFP MN cultures treated with VX-680 and ZM447439 (FIG. 4A).

### AKIs Decrease the Toxic Effect of SOD1^{G93A} Astrocytes

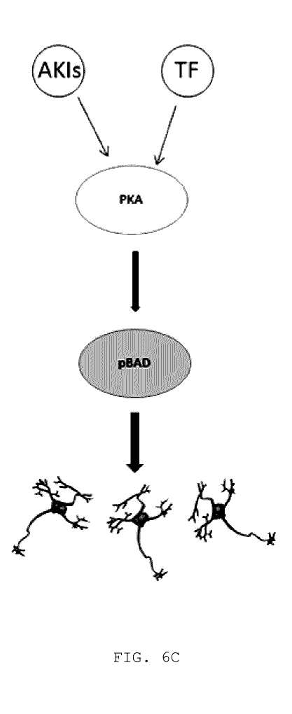

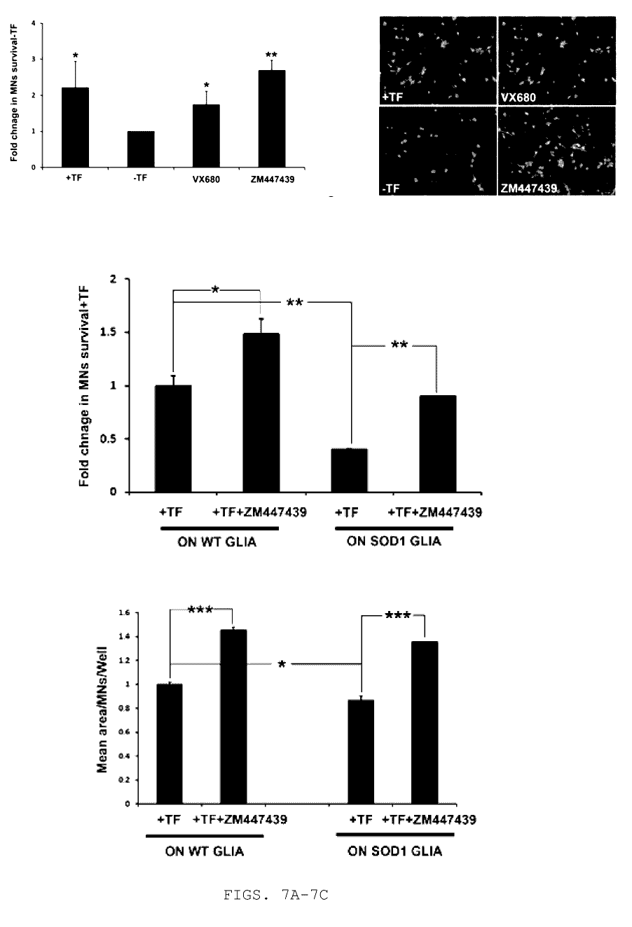

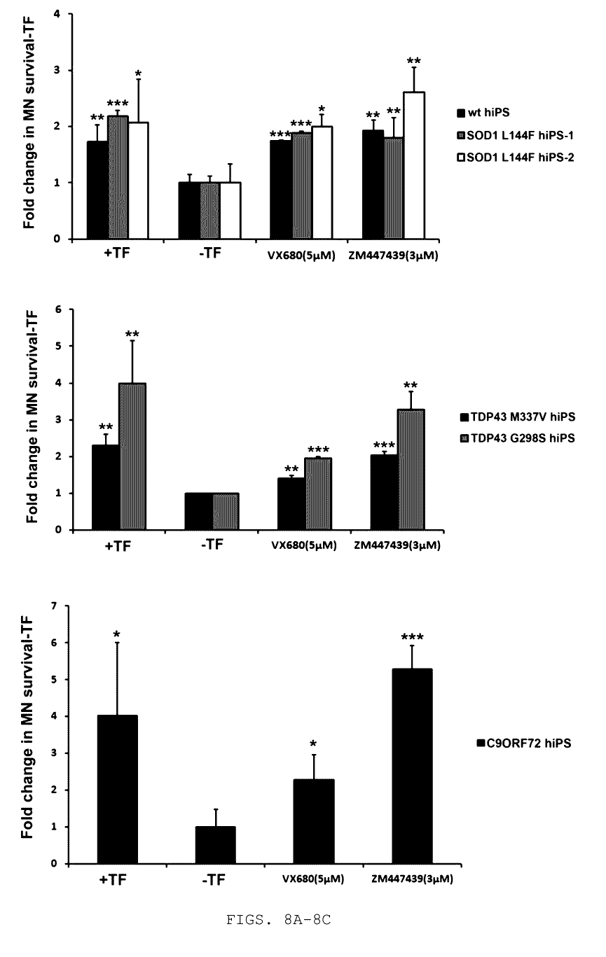

SOD1^{G93A} mutant astrocytes have been shown to have toxic effects on MNs in a co-cultures (Di Giorgio et al., 2007). We sought to examine whether AKIs decrease the toxic effect of SOD1^{G93A} mutant astrocytes. We first cultured primary astrocytes from wild type (WT) and SOD1^{G93A} mice and plated FACS purified MNs on top. We observed significant cell death when MNs were cultured on mutated astrocytes compared to WT astrocytes. ZM447439 improved both Hb9::GFP and SOD1^{G93A}/Hb9::GFP MN survival. We observed less of an increase in survival when SOD1^{G93A}/Hb9::GFP MN were co-cultured with SOD1^{G93A} astrocytes, which could be due to accumulated toxicity of mutant MNs and mutant astrocytes. using size as an indicator of MN health, we analyzed the area of MN cell bodies and found that it was increased in the presence of ZM447439 (Supp FIG. 7B).

### AKIs Promote MN Survival Through the PKA Pathway

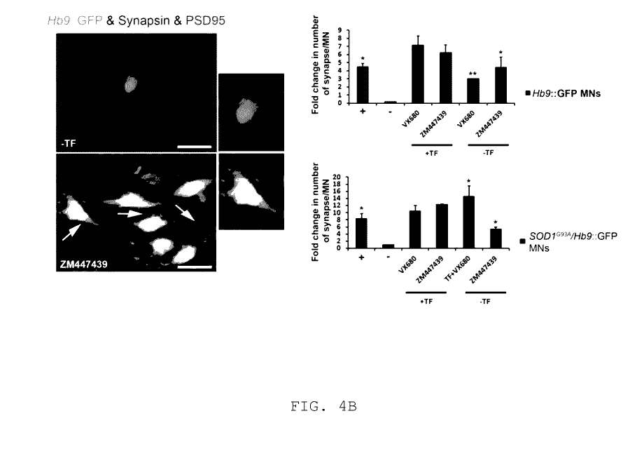

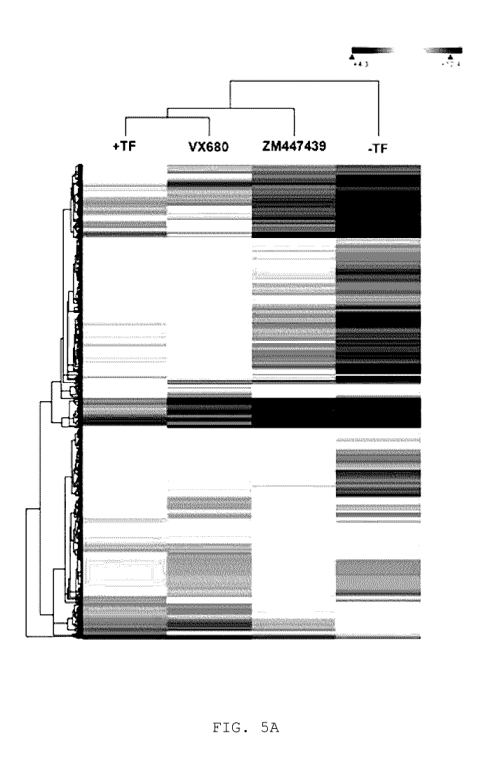

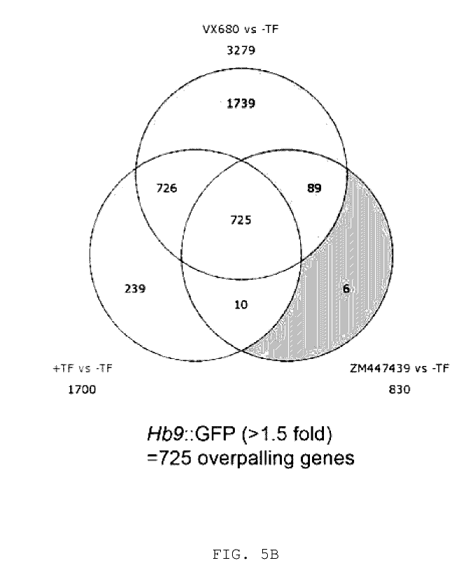

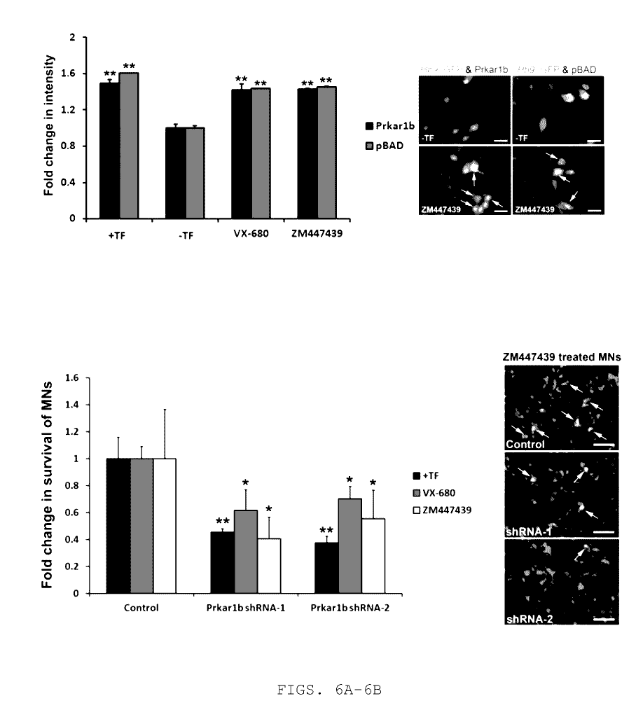

Next we sought to investigate the mechanism by which AKIs promote MN survival. To evaluate this comprehensively we performed microarray analysis to compare gene expression between MNs cultured with: 1) Trophic factors (+TF), 2) No trophic factors (−TF), 3) VX-680 in the absence of TFs, and 4) ZM447439 in the absence of TFs. Microarray data was validated by qPCR for selected up-regulated and down-regulated genes (FIG. 5C). Heat maps generated showed a similar gene expression pattern in +TF, VX-680 and ZM447439 treated MNs as they were clustered together and separate from −TF (FIG. 5A). Venn diagram analysis was performed to look for overlapping candidate genes among experimental conditions whose expression was significantly changed. Gene expression was compared between +TF and −TF, VX-680 and −TF, and ZM447439 and −TF. With a cut off of more than 3 fold difference, there were 85 genes overlapping in all three comparison in Hb9::GFP MNs and 57 in SOD1^{G93A}/Hb9::GFP MNs. We found 39 overlapping genes when we compared both types of MNs (FIG. 5B). There were numerous genes related to synapse formation (Syt1, Snap25 and Sv2c, Map2 and Dcx) whose expression was upregulated in the +TF, VX-680 & ZM447439 conditions, re-confirming our previous results. We analyzed the shortlist of 39 genes by using DAVID functional annotation bioinformatics microarray analysis software to find the potential pathways involved in MN survival. Based on increased expression of Prkar1b, PKA pathway activation was identified as a candidate for promoting MN survival. PKA is known to phosphorylate BAD protein, leading to increased cell survival (Jose M. LIZCANO et al., 2000; Julian Downward et al., Nature cell biology 1999; Komaki S et al., Neurosci Lett 2012). We confirmed increased levels of Prkarlb and phosphorylated BAD (pBAD) by immunostaining in surviving MNs in the presence of trophic factors, or in VX-680 & ZM447439 treated cultures (FIG. 6A). We further investigated this pathway by lentivirus knockdown of Prkarlb. Knocking down Prkarlb effectively decreased MN survival in the presence of VX-680, ZM447439 or trophic factors (FIG. 6B). Together, these results indicate that AKIs exert an MN survival effect by activating the PKA pathway, effectively blocking apoptosis in MNs (FIG. 6C).

AKIs promote survival of MNs derived from human ESCs and ALS iPSCs

For AKIs to qualify as ALS therapeutic candidates, it is important to demonstrate that they promote survival of human MNs in addition to mouse cells. We generated MNs from a human ESC line expressing an HB9::GFP transgene [HuES-3/Hb9::GFP (Di Giorgio et al., 2008)]. Human ESC derived MN cultures were pre-treated with AraC to eliminate progenitor cells. We induced MN death by again withdrawing trophic factors. We found that VX-680 and ZM447439 treatment rescued human MN death. We again found ZM447439 to be more effective and potent in preventing MN death (FIG. 7A). We also investigated the toxic effect of SOD1^{G93A} astrocytes on human ESC derived MN survival. We found significant death of human MNs cultured on mutant astrocytes. This toxicity induced death was completely rescued by ZM447439 (FIG. 7B). As described above, we analyzed the size of human MN co-cultured with both wild-type and mutant astrocytes and found that it was significantly increased in the presence of ZM447439 (FIG. 7C).

We produced MNs from human iPSCs lines including a healthy control, two ALS patients expressing confirmed mutations in SOD1 (Boulting et al., 2011), two with mutations in TDP-43 and one in C90RF72. We treated these cultures with AraC to minimize the number of proliferating progenitors. In all human iPSC derived MN cultures, VX-680 and ZM447439 were able to substantially increase MN survival (FIGS. 8A, 8B & 8C). ZM447439 was again more effective and potent in rescuing human MN death. We also examined the effect of SOD 1^{G93A} astrocytes co-culture on iPSC derived MNs. We found no toxic effect on survival in any of the patient iPSC derived MNs, but we did observe decreased MN survival in the control iPSC derived MNs. ZM447439 improved survival in all of the iPSCs derived MNs cultured on wild type or SOD1^{G93A} astrocytes. We also looked at the effect of AKIs on morphology and synapse formation. We found that ZM447439 preserved both in all iPSC-derived MN lines in the absence of trophic factor. In addition, by immunoblot we observed that ZM447439 enhanced synaptic protein, synaptophysin, expression in both the presence and absence of trophic factors.

### Discussion

Although ALS is the most common MN disease in adults, there are currently no truly effective treatments for it. While better treatments for ALS are urgently needed, it has been challenging to conduct research geared towards therapeutic discovery, partly because of the diverse causes of ALS, a mostly idiopathic disorder. High-throughput screening (HTS) of chemical libraries has become a critical tool in basic biology and drug discovery. Recent studies have shown a strong increase in the discovery of first-in-class drugs arising from phenotypic screening methodologies. Although primary cells would be the ideal cell type for phenotypic screening, the isolation of primary MNs is extremely difficult, making high throughput screening almost impossible. Stem cells, on the other hand, have a unique ability to both continually self-renew as well as to differentiate into specialized cells of any lineage and thus could provide a continuous and large supply of specialized cells to perform screens that may lead to the discovery of new drugs. Fortunately, there are now simple protocols available for differentiating mouse and human embryonic stem cells (ESCs) and iPSCs into MNs. Recently, a high content small molecule screen performed using mESCs derived MNs identified Kenpaullone as a potential ALS therapeutic (Yang et al., 2013). In another study, Hoing et al (2012) demonstrated that a screening assay using stem cell derived MN co-cultured with microglia cell line could be used to identify candidate therapeutics. These studies strongly support the concept of carrying out small molecule screens on stem cell derived MNs. In the present study, we have carried out a survival screen to test a library of 300 small molecule kinase inhibitors. We performed this screen using wild-type and SOD1^{G93A} MNs in order to identify hits that could protect MNs broadly, thus creating an opportunity to identify a lead molecule that could be therapeutic for various form of ALS. AKIs were identified as the most robust in promoting MN survival under various conditions, remarkably maintaining cell integrity and synapse formation.

Previous work has shown that astrocytes from SOD1^{G93A} mice are toxic to MNs (Di Giorgio et al., 2008). AKIs protect MNs from mutant astrocytes-induced death in a co-culture environment. This survival effect was not observed when SOD1^{G93A} MNs were cultured with mutant astrocytes. This may be due to accumulated toxicity of the mutated SOD1^{G93A} protein which cannot be prevented by presence of the AKI. Our data suggest that AKIs can protect MNs not only from growth factor withdrawal induced death but can also promote survival when death is caused by other type of stimuli.

Aurora kinases are serine/threonine kinases that are essential for cell proliferation (Zhou H et al., 1998; Sen S et al., 2002). Both the expression level and the kinase activity of Aurora kinases are known to be up-regulated in many types of human cancer. Apart from their role in mitosis Aurora Kinase A has been shown to mediate the phosphorylation of the polarity complex protein Par3 and regulate its function in neuronal polarity (Khazaei et al., 2009). Aurora Kinase has also been shown to form a PKC-Aurora A-NDEL 1 complex that regulates neurite elongation by modulating microtubule dynamics (Mori et al., 2009). To date a direct role of Aurora Kinases in cell survival has never been reported. The current study opens a new avenue of intrigue into their function.

Microarray from AKIs treated MNs showed increased expression of numerous synaptic and neural cytoskeletal genes, validate previous results that MNs in presence of AKIs not only survive better but also preserve their morphology and make more synapses. Our data suggest that AKIs exert a cell survival effect by activating the PKA pathways, already well known for its role in cell survival (Julian et al., 1999; Jose et al., 2000; Komaki et al., 2012). Thus, activation of cAMP-PKA pathway could also be a potential target for treating ALS disease. Microarray data also showed decreased expression of Leftyl and Lefty 2 genes, a member of TGF-beta family, in AKIs treated MNs. TGF-beta signaling has been shown to be inhibited during MN differentiation (Ichida et al., 2009). It will be interesting to further investigate the role of Lefty genes in MN survival.

For AKIs to be relevant to treating ALS, it is important to demonstrate that they are effective in promoting the survival of human MNs. We have demonstrated that AKIs support survival for an array of human ESCs and iPSCs derived motor neurons, including when co-cultured with WT or mutant astrocytes. The next step would be to test these drug candidates in animal ALS models, but in order to do this it is important to make them as potent as possible and able to penetrate into the spinal cord. It will be interesting to investigate whether AKIs could improve disease phenotype in ALS mouse models. To conclude, we report that Aurora Kinase Inhibitors (AKIs), especially ZM447439, emerges as compound of great interest as potential ALS therapeutics.

What is claimed is:

1. A method of promoting motor neuron survival, comprising contacting a motor neuron or population of cells comprising a motor neuron with an effective amount of an agent that inhibits Aurora kinase.

2. A method according to claim 1, wherein the agent increases activation of the anti-apoptotic protein kinase A pathway.

3. A method according to any one of claim 1 or 2, wherein the agent increases phosphorylation of a protein in the anti-apoptotic protein kinase A pathway.

4. A method according to claim 3, wherein the protein is Bc1-2-associated death promoter (BAD).

5. A method according to any one of claims 1-4, wherein the agent is a pan Aurora kinase inhibitor.

6. A method according to any one of claims 1-5, wherein the agent inhibits Aurora kinase A.

7. A method according to any one of claims 1-6, wherein the agent inhibits Aurora kinase B.

8. A method according to any one of claims 1-7, wherein the agent inhibits Aurora kinase C.

9. A method according to any one of claims 1-8, wherein the agent is selected from the group consisting of VX-608, ZM447439, 4-4-Ben, MLN8054, PHA-680632, TAK-901, AMG900, PF-03814735, CCT129202, phtalazinonepyrazole, hesperidin hydrochloride, CCT 137690, TC-A 2317 hydrochloride, Aurora kinase inhibitor II, JNJ-7706621, H-1152, PHA739358, OM137, SNS-314, AT9283, CYC-116, MLN8237, ENMD-2076, SBE 13 hydrochloride, analogs or derivatives thereof, and combinations thereof.

10. A method according to any one of claims 1-9, wherein the agent is selected from the group consisting of small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

11. A method according to any one of claims 1-10, wherein the motor neuron are selected from the group consisting of a HB9 motor neuron, a G93A motor neuron, a HB9(WT-SOD1) motor neuron, a HUES3 derived motor neuron, and combinations thereof.

12. A method according to any one of claims 1-11, wherein the motor neuron comprises a mutation in a gene encoding superoxide dismutase 1 (SOD1).

13. A method according to claim 12, wherein the mutation is a G93A mutation.

14. A method according to any one of claims 1-13, wherein the contact is in vitro or ex vivo.

15. A method of treating or preventing a neurodegenerative disorder in a subject in need thereof, comprising administering to the subject an effective amount of an agent that inhibits Aurora kinase.

16. A method according to claim 15, wherein the agent increases activation of the anti-apoptotic protein kinase A pathway.

17. A method according to any one of claim 15 or 16, wherein the agent increases phosphorylation of a protein in the anti-apoptotic protein kinase A pathway.

18. A method according to claim 17, wherein the protein is Bc1-2-associated death promoter (BAD).

19. A method according to any one of claims 15-18, wherein the agent is a pan Aurora kinase inhibitor.

20. A method according to any one of claims 15-19, wherein the agent inhibits Aurora kinase A.

21. A method according to any one of claims 15-20, wherein the agent inhibits Aurora kinase B.

22. A method according to any one of claims 15-21, wherein the agent inhibits Aurora kinase C.

23. A method according to any one of claims 15-22, wherein the agent is selected from the group consisting of VX-608, ZM447439, 4-4-Ben, MLN8054, PHA-680632, TAK-901, AMG900, PF-03814735, CCT129202, phtalazinonepyrazole, hesperidin hydrochloride, CCT 137690, TC-A 2317 hydrochloride, Aurora kinase inhibitor II, JNJ-7706621, H-1152, PHA739358, OM137, SNS-314, AT9283, CYC-116, MLN8237, ENMD-2076, SBE 13 hydrochloride, analogs or derivatives thereof, and combinations thereof.

24. A method according to any one of claims 15-23, wherein the agent is selected from the group consisting of small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

25. A method according to any one of claims 15-24, wherein the subject selected for treatment of a neurodegenerative disorder or disorder characterized by neuronal cell death.

26. A method according to any one of claims 15-25, wherein the subject is at risk of developing a neurodegenerative disorder or a disorder characterized by neuronal cell death.

27. A method according to any one of claims 15-26, wherein the subject is suspected of having a neurodegenerative disorder or a disorder characterized by neuronal cell death.

28. A method according to any one of claims 15-27, wherein the subject is a mammal.

29. A method according to any one of claims 15-28, wherein the subject is a human.

30. A method according to any one of claims 15-29, wherein the neurodegenerative disorder is characterized by mutation of a SOD gene.

31. A method according to any one of claims 15-30, wherein the neurodegenerative disorder is characterized by decreased levels of SOD protein.

32. A method according to any one of claims 15-31, wherein the neurodegenerative disorder is characterized by neuronal cell death.

33. A method according to any one of claims 15-32, wherein the neurodegenerative disorder is ALS.

34. A method of identifying a candidate agent that promotes motor neuron survival, comprising (a) contacting a population of cells comprising motor neurons with a test agent, and (b) measuring (i) the level or activity of an Aurora kinase or (ii) activation of the anti-apoptotic protein kinase A pathway, in the presence of the test agent, and (c) identifying the candidate agent that promotes motor neuron survival, wherein the test agent is a candidate agent for promoting motor neuron survival if the test agent (i) decreases the level or activity of the Aurora kinase or (ii) increases activation of the anti-apoptotic protein kinase A pathway, in the presence of the test agent.

35. A method of identifying a candidate agent for treating or preventing a neurodegenerative disorder, comprising (a) contacting a population of cells comprising motor neurons with a test agent, and (b) measuring (i) the level or activity of an Aurora kinase or (ii) activation of the anti-apoptotic protein kinase A pathway, in the presence of the test agent, and (c) identifying the candidate agent for treating or preventing a neurodegenerative disorder, wherein the test agent is a candidate agent for treating or preventing a neurodegenerative disorder if the test agent (i) decreases the level or activity of the Aurora kinase or (ii) increases activation of the anti-apoptotic protein kinase A pathway, in the presence of the test agent.

36. A method according to claim 35, wherein the neurodegenerative disorder is amyotrophic lateral sclerosis.

37. A method according to any one of claims 34-36, wherein the population of cells comprises glial cells.

38. A method according to any one of claims 34-37, wherein the contacting is performed in the absence of trophic factors.

39. A method according to any one of claims 34-38, wherein the motor neurons are selected from the group consisting of a HB9 motor neuron, a G93A motor neuron, a HB9(WT-SOD1) motor neuron, a HUES3 derived motor neuron, and combinations thereof.

40. A method according to any one of claims 34-39, wherein the motor neuron comprises a mutation in a gene encoding superoxide dismutase 1 (SOD1).

41. A method according to claim 40, wherein the mutation is a G93A mutation.

42. A method according to any one of claims 34-41, wherein the motor neuron comprises an in vitro-differentiated motor neuron.

43. A method according to any one of claims 34-42, wherein the motor neurons are derived from pluripotent cells selected from the group consisting of embryonic stem cells (ESCs) and induced pluripotent stem cells (iPSCs).

44. A method according to any one of claims 34-43, wherein the motor neurons are derived from an individual suffering from, diagnosed with, or at risk of developing ALS.

45. A method according to any one of claims 34-44, wherein the motor neurons comprise human motor neurons.

46. A method according to any one of claims 34-45, wherein the test agent is selected from the group consisting of small organic or inorganic molecules; saccharides; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

47. A method according to any one of claims 34-46, further comprising quantifying the number of motor neurons surviving in the presence of the test agent.

48. A method according to claim 47, wherein the surviving motor neurons express a detectable reporter.

49. A method according to claim 48, wherein the detectable reporter is a fluorescent protein selected from the group consisting of green fluorescent protein (GFP) and red fluorescent protein (RFP).

50. A method for diagnosing a neurodegenerative disorder in a subject, the method comprising: (a) obtaining a biological sample comprising neuronal cells from the subject; (b) conducting at least one assay on the neuronal cells in the biological sample to detect the level or activity of Aurora kinase in the neuronal cells; and (c) diagnosing the subject as having a neurodegenerative disorder if the level or activity of the Aurora kinase in the neuronal cells is increased relative to a level or activity of Aurora kinase in a control sample.

51. A method for predicting the progression of a neurodegenerative disorder in a subject, the method comprising: (a) obtaining a first biological sample comprising neuronal cells from a subject diagnosed as having a neurodegenerative disorder; (b) obtaining a second biological sample comprising neuronal cells from the subject at a time which is later than when the first biological sample was obtained; (c) conducting at least one assay on the neuronal cells in the biological samples to detect a level or activity of Aurora kinase in the neuronal cells; and (d) predicting the progression of the neurodegenerative disorder in the subject, wherein: (i) the neurodegenerative disorder is predicted to progress if the level or activity of Aurora kinase in the neuronal cells in the second biological sample is increased relative to the level or activity of Aurora kinase in in the first biological sample; or (ii) the neurodegenerative disorder is not predicted to progress if the level or activity of Aurora kinase in the neuronal cells in the second biological sample is decreased relative to the level or activity of Aurora kinase in in the first biological sample.

52. A method of monitoring the effectiveness of a therapy in reducing the progression of a neurodegenerative disorder in a subject, the method comprising: (a) conducting at least one assay to determine the level or activity of Aurora kinase in a biological sample comprising neuronal cells from a subject having a neurodegenerative disorder prior to and following administration of the therapy to the subject; and (b) comparing the level or activity of Aurora kinase in the biological sample from the subject prior to the administration of the therapy to the level or activity of Aurora kinase in the biological sample from the subject following administration of the therapy; and (c) monitoring the effectiveness of the therapy in reducing the progression of the neurodegenerative disorder in the subject, wherein a decrease in the level or activity of Aurora kinase in the biological sample following administration of the therapy as compared to the level or activity of Aurora kinase in the biological sample prior to the administration of the therapy is an indication that the therapy is effective in reducing the progression of the neurodegenerative disorder in the subject.

53. The method of any one of claims 50-52, wherein the at least one assay comprises a hybridization assay to detect the expression of Aurora kinase.

54. The method of claim 53, wherein the hybridization assay is selected from the group consisting of a microarray and qRT-PCR.

55. The method of any one of claims 50-52, wherein the at least one assay comprises a sequencing assay to detect the expression of Aurora kinase.

56. The method of claim 55, wherein the sequencing assay is selected from the group consisting of serial analysis of gene expression (SAGE), cap analysis of gene expression (CAGE), massively parallel signature sequencing (MPSS), GRO-seq, and RNA-seq.

57. The method of any one of claims 50-52, wherein the at least one assay comprises immunostaining to detect Aurora kinase protein levels.

58. The method of claim 57, wherein the immunostaining is selected from the group consisting of Western blot, ELISA, and flow cytometry.

59. The method of any one of claims 50-52, wherein the at least one assay comprises a phosphorylation assay to detect phosphorylation of Aurora kinase.

60. The method of claim 59, wherein the at least one assay comprises a phosphorylation assay to detect phosphorylation of Aurora kinase at threonine 288 (T288) or serine 331 (S331).

61. The method of any one of claims 50-52, wherein the at least one assay comprises a phosphorylation assay to detect the phosphorylation activity of Aurora kinase.

62. The method of claim 61, wherein the at least one assay comprises a protein kinase assay to detect the level of phosphorylation of a protein in the anti-apoptotic protein kinase A pathway.

63. The method of claim 61 or 62, wherein the at least one assay comprises a protein kinase assay to detect the level of phosphorylation of BAD protein.

64. The method of any one of claims 50-63, further comprising selecting a subject suspected of having a neurodegenerative disorder.

65. The method of any one of claims 50-64, wherein the neuronal cells comprise motor neurons.

66. The method of any one of claims 50-65, wherein the neuronal cells comprise sensory neurons.

67. The method of any one of claims 50-66, wherein the neurodegenerative disorder is ALS.

68. The method according to any one of claims 50-67, wherein the Aurora kinase is selected from Aurora kinase A, Aurora kinase B, Aurora kinase C, and combinations thereof.

69. The method of claim 52, wherein the therapy comprises an agent that inhibits Aurora kinase selected from a pan Aurora kinase inhibitor, an inhibitor of Aurora kinase A, an inhibitor of Aurora kinase B, and an inhibitor of Aurora kinase C.
